(19) United States
(12) Patent Application Publication
(10) Pub. No.: US 2012/0100989 A1
(43) Pub. Date: Apr. 26, 2012

(54) 3-Aryl-4-(2-thienylmethylene)isoxazol-5(4H)-ones as Fungicides

(75) Inventors: Werner HALLENBACH, Monheim (DE); Oliver Guth, Leverkusen (DE); Thomas Seitz, Langenfeld (DE); Heinz-Juergen Wroblowsky, Langenfeld (DE); Philippe Desbordes, Lyon (FR); Ulrike Wachendorff-Neumann, Neuwied (DE); Peter Dahmen, Neuss (DE); Arnd Voerste, Koln (DE); Peter Lösel, Leverkusen (DE); Olga Malsam, Rosrath (DE); Rachel Rama, Saint-Pierre-Ia-Palud (FR); Hiroyuki Hadano, Tochigi (JP)

(73) Assignee: Bayer CropScience AG, Monheim (DE)

(21) Appl. No.: 13/165,968

(22) Filed: Jun. 22, 2011

Related U.S. Application Data
(60) Provisional application No. 61/357,764, filed on Jun. 23, 2010.

(30) Foreign Application Priority Data
Jun. 22, 2010 (EP) 10166826.7

(51) Int. Cl. A01N 43/80; A01P 3/00; A01P 13/00; A01P 1/00; A01P 7/02; A01P 5/00; C07D 413/06; A01P 7/04

(52) U.S. Cl. 504/101; 548/243; 514/380; 504/358

(57) ABSTRACT

The invention relates to 3-aryl-4-(2-thienylmethylene)isoxazol-5(4H)-ones, to agrochemically active salts thereof, to their use and to methods and compositions for controlling phytopathogenic harmful fungi and insects in and/or on plants or in and/or on seed of plants, to processes for preparing such compositions and to treated seed, and to their use for controlling phytopathogenic harmful fungi in agriculture, horticulture and forestry, in animal health, in the protection of materials and in the domestic and hygiene field. The present invention further relates to a process for preparing isoxazolone derivatives.

The invention relates to 3-aryl-4-(2-thienylmethylene)isoxazol-5(4H)-ones, to agrochemically active salts thereof, to their use and to methods and compositions for controlling phytopathogenic harmful fungi and insects in and/or on plants or in and/or on seed of plants, to processes for preparing such compositions and to treated seed, and to their use for controlling phytopathogenic harmful fungi in agriculture, horticulture and forestry, in animal health, in the protection of materials and in the domestic and hygiene field. The present invention further relates to a process for preparing isoxazolone derivatives.

It is already known that certain 4-ylideneisoxazol-5(4H)-ones can be used as fungicidal crop protection agents. Thus, the synthesis of various 4-arylideneisoxazol-5(4H)-ones and their biological action against various fungal diseases such as, for example, Erysiphe graminis, Plasmopara viticola, Phytophthora infestans, Pyricularia oryzae and Fusarium culmorum has been described (GB-A 1,074,803). However, in particular at relatively low application rates, the fungicidal activity of these compounds is not always sufficient. The preparation of 4-arylideneisoxazol-5(4H)-ones and their fungicidal action are also described in J. Ind. Chem. Soc. 1977, 54, 485. However, the examples shown in this publication are limited to 3-methylarylideneisoxazol-5(4H)-ones whose fungicidal activity at low application rates is likewise not always sufficient. Finally, the patent specification DD 54366 describes the preparation of 3-substituted 3-chloro-2-propen-1-ylideneisoxazol-5(4H)-ones. However, this publication does not mention any fungicidal activity.

Since the ecological and economical demands made on modem active compounds, for example fungicides and insecticides, are increasing constantly, for example with respect to activity spectrum, toxicity, selectivity, application rate, formation of residues and favourable manufacture, and there can furthermore be problems, for example, with resistances, there is a constant need to develop novel fungicidal and insecticidal compositions which, at least in some areas, have advantages over the known ones.

Surprisingly, it has now been found the present 3-aryl-4-[2-(thiophen-2-yl)ethenyl]isoxazol-5(4H)-ones achieve at least some aspects of the objects mentioned and are therefore suitable for use as fungicides and insecticides. From the compounds described in GB-A 1,074,803, the present 3-aryl-4-(2-thienylmethylene)-isoxazol-5(4H)-ones are distinguished by their surprisingly high activity even at low application rates.

The invention relates to compounds of the formula (I)

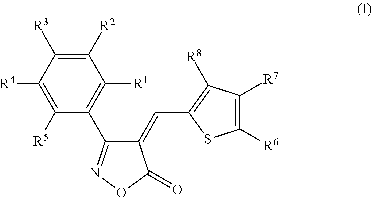

in which
(1) group 1:
R¹ represents A, but not hydrogen, hydroxyl or methoxy,
R² to R⁵ independently of one another represent A, where furthermore in each case two adjacent radicals R¹ to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ to R⁸ independently of one another represent A,
   or
   (2) group 2:
R¹ represents hydrogen or methoxy,
R² represents A, but not hydrogen, methoxy, nitro or dimethylcarbamothioylamino [NHC(S)NMe₂],
R³ to R⁵ independently of one another represent A, where furthermore in each case two adjacent radicals R² to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ to R⁸ independently of one another represent A,
   or
   (3) group 3:
R¹ represents hydrogen or methoxy,
R² represents hydrogen, methoxy, nitro or dimethylcarbamothioylamino [NHC(S)NMe₂],
R³ represents A, but not hydrogen, nitro, C₁-C₃-alkoxy, amino, CO₂H or methyl,
R⁴ to R⁵ independently of one another represent A, where furthermore in each case two adjacent radicals R³ to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ to R⁸ independently of one another represent A,
   or
   (4) group 4:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ to R⁵ independently of one another represent A, where furthermore R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents A, but not hydrogen, nitro, dialkylamino, C₁-C₈-alkyl, hetaryl or morpholin-4-yl,
R⁷ and R⁸ independently of one another represent A,
   or
   (5) group 5:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ to R⁵ independently of one another represent A, where furthermore the radicals R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents hydrogen, nitro, dialkylamino, C₁-C₈-alkyl, hetaryl or morpholin-4-yl,
R⁷ represents A, but not hydrogen, bromine or nitro,
R⁸ represents A,
   or
   (6) group 6:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ and R⁵ independently of one another represent A, where furthermore R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents hydrogen, nitro, dialkylamino, C₁-C₈-alkyl, hetaryl or morpholin-4-yl,
R⁷ represents hydrogen, bromine or nitro,
R⁸ represents A, but not hydrogen or methyl,
   and
A represents hydrogen, halogen, cyano, nitro, OR⁹, SR⁹, SOR⁹, SO₂R⁹, SO₂NR⁹R¹⁰, COR⁹, C═N—OR⁹, CSR⁹, NR⁹CO₂R¹⁰, NR⁹C(O)SR¹⁰, NR⁹C(S)OR¹⁰, NR⁹R¹⁰, NR⁹COR¹⁰, NR⁹CSR¹⁰, NR⁹SO₂R¹⁰, OCONR⁹R¹⁰, OCSNR⁹R¹⁰, NR⁹C(O)NR⁹R¹⁰, NR⁹C(S)NR⁹R¹⁰, O(CO)R⁹, O(CS)R⁹, CONR⁹R¹⁰, CSNR⁹R¹⁰, CO₂R⁹, C(O)SR⁹, C(S)OR⁹, (CH₂)ₘOR⁹, (CH₂)ₘSR⁹, (CH₂)ₘNR⁹R¹⁰, (CH₂)ₘCO₂R¹⁰, (CH₂)ₘNR⁹CO₂R¹⁰, C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl, substituted or unsubstituted phenyl or hetaryl or heterocyclyl, substituted or unsubstituted benzyl or —CH₂-hetaryl,
R⁹ and R¹⁰ independently of one another represent hydrogen, unsubstituted or substituted C₁-C₈-alkyl, C₂-C₈-alkenyl, C₂-C₈-alkynyl, C₁-C₈-haloalkyl, C₃-C₈-cycloalkyl, tri-C₁-C₄-alkyl-silyl, in each case substituted or unsubstituted aryl, —CH₂-aryl, hetaryl or —CH₂-hetaryl or heterocyclyl, where the two radicals R⁹ and R¹⁰ together with this grouping to which they are attached form a 3- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
   m represents the number 1, 2, 3, 4, 5, 6, 7 or 8,
   to their agrochemically active salts and also to their use for controlling unwanted microorganisms and insects in crop protection and in the protection of materials.

The invention furthermore provides the use of compounds of the formula (I), group U, in which
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ and R⁵ independently of one another represent A, where furthermore R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents hydrogen, nitro, dialkylamino, C₁-C₈-alkyl, hetaryl or morpholin-4-yl,
R⁷ represents hydrogen, bromine or nitro,
R⁸ represents hydrogen or methyl,
A represents hydrogen, halogen, cyano, nitro, OR⁹, SR⁹, SOR⁹, SO₂R⁹, SO₂NR⁹R¹⁰, COR⁹, C═N—OR⁹, CSR⁹, NR⁹CO₂R¹⁰, NR⁹C(O)SR¹⁰, NR⁹C(S)OR¹⁰, NR⁹R¹⁰, NR⁹COR¹⁰, NR⁹CSR¹⁰, NR⁹SO₂R¹⁰, OCONR⁹R¹⁰, OCSNR⁹R¹⁰, NR⁹C(O)NR⁹R¹⁰, NR⁹C(S)NR⁹R¹⁰, O(CO)R⁹, O(CS)R⁹, CONR⁹R¹⁰, CSNR⁹R¹⁰, CO₂R⁹, C(O)SR⁹, C(S)OR⁹, (CH₂)ₘOR⁹, (CH₂)ₘSR⁹, (CH₂)ₘNR⁹R¹⁰, (CH₂)ₘCO₂R¹⁰, (CH₂)ₘNR⁹CO₂R¹⁰, C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl, substituted or unsubstituted phenyl or hetaryl or heterocyclyl, substituted or unsubstituted benzyl or —CH₂-hetaryl,
R⁹ and R¹⁰ independently of one another represent hydrogen, unsubstituted or substituted C₁-C₈-alkyl, C₁-C₈-alkenyl, C₂-C₈-alkynyl, C₁-C₈-haloalkyl, C₃-C₈-cycloalkyl, tri-C₁-C₄-alkyl-silyl, in each case substituted or unsubstituted aryl, —CH₂-aryl, hetaryl or —CH₂-hetaryl or heterocyclyl, where the two radicals R⁹ and R¹⁰ together with this grouping to which they are attached form a 3- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
   m represents the number 1, 2, 3, 4, 5, 6, 7 or 8,
   as fungicides and insecticides.

The formula (I) provides a general definition of the 3-aryl-4-(2-thienylmethylene)isoxazol-5(4H)-ones according to the invention. Preferred radical definitions are stated below.
A preferably represents hydrogen, fluorine, chlorine, bromine, iodine, cyano, nitro, OR⁹, SR⁹, SOR⁹, SO₂R⁹, SO₂NR⁹R¹⁰, COR⁹, NR⁹CO₂R¹⁰, NR⁹R¹⁰, NR⁹COR¹⁰, NR⁹SO₂R¹⁰, OCONR⁹R¹⁰, NR⁹C(O)NR⁹R¹⁰, NR⁹C(S)NR⁹R¹⁰, O(CO)R⁹, CONR⁹R¹⁰, CO₂R⁹, (CH₂)ₘOR⁹, (CH₂)ₘSR⁹, (CH₂)ₘN(R⁹R¹⁰)₂, (CH₂)ₘCO₂R⁹, (CH₂)ₘNR⁹CO₂O, C₁-C₆-alkyl, C₂-C₆-alkenyl, C₂-C₆-alkenyl, C₁-C₆-haloalkyl having in each case 1 to 13 fluorine, chlorine and/or bromine atoms, C₃-C₆-cycloalkyl, C₃-C₆-cycloalkenyl, tri-C₁-C₄-alkyl-silyl, phenyl-(di-C₁-C₄-alkyl)-silyl, phenyl, benzyl, pyridinyl, pyridinylmethyl, thienyl, thenyl, furyl, furfuryl, pyrrolyl, pyrrolylmethyl or represents phenyl, benzyl, pyridinyl, pyridinylmethyl, thienyl, thenyl, furyl, furfuryl, pyrrolyl, pyrrolylmethyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms.
A particularly preferably represents hydrogen, fluorine, chlorine, bromine, cyano, hydroxyl, methoxy, ethoxy, n-propoxy, isopropoxy, n-, s-, t-butoxy, —O(CH₂)₂OH, —O(CH₂)₂OCH₃, —O(CH₂)₃OH, —O(CH₂)₃OCH₃, trifluoromethoxy, trichloromethoxy, SH, S-Me, S-Et, S—Pr, S-iPr, S-Bu, S-secBu, S-isoBu, S-tBu, SCF₃, SO-Me, SO-Et, SO—Pr, SO-iPr, SO-Bu, SO-secBu, SO-isoBu, SO-tBu, SO₂-Me, SO₂-Et, SO₂—Pr, SO₂-iPr, SO₂—Bu, SO₂-secBu, SO₂-isoBu, SO₂-tBu, SONHMe, SONMe₂, SONHEt, SONEt₂, SONHPr, SONPr₂, SONHBu, SONBu₂, SONHCF₃, SON(CF₃)₂, SO₂NHMe, SO₂NMe₂, SO₂NEt₂, SO₂NHEt, SO₂NPr₂, SO₂NHPr, SO₂NHCF₃, SO₂N(CF₃)₂, COMe, COEt, COPr, COiPr, COBu, COsecBu, COisoBu, COtBu, COCF₃, NHCO₂Me, NHCO₂Et, NHCO₂Pr, NHCO₂iPr, NHCO₂Bu, NHCO₂secBu, NHCO₂isoBu, NHCO₂tBu, NHCOMe, NHCOEt, NHCOPr, NHCOiPr, NHCOBu, NHCOiBu, NHCOsecBu, NHCOisoBu, NHCOtBu, NHCO(CH₂)₂OH, NHCO(CH₂)₂OCH₃, NHCO(CH₂)₃OH, NHCO(CH₂)₃CCH₃, N(Me)COMe, N(Me)COEt, N(Me)COPr, N(Me)COiPr, N(Me)COBu, N(Me)COsecBu, N(Me)COtBu, N(Me)CO(CH₂)₂OH, N(Me)CO(CH₂)₂OCH₃, N(Me)CO(CH₂)₃OH, N(Me)CO(CH₂)₃OCH₃, NMe₂, NEt₂, NHMe, NH₂, NHtBu, NHEt, NHPr, NHBu, NHiBu, NHsecBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHPr, OCONHiPr, OCONHBu, OCONHsecBu, OCONHisoBu, OCONHtBu, OCONMe₂, OCONEt₂, OCONFr₂, OCONiPr₂, OCONBu₂, OCONsecBu₂, OCONisoBu₂, OCONHtBu₂, NHC(O)NH₂, NHC(O)NMe₂, NMeC(O)NH₂, NMeC(O)NMe₂, NHC(S)NH₂, NHC(S)NMe₂, NMeC(S)NH₂, NMeC(S)NMe₂, OCOMe, OCOEt, OCOPr, OCOIPr, OCOBu, OCOsecBu, OCOisoBu, OCOtBu, CONHEt, CONEt₂, CONHMe, CONMe₂, CONHPr, CONPr₂, CONHBu, CONHsecBu, CONHisoBu, CONHtBu, CONHCH(CH₃)CH₂OH, CONHCH(CH₃)CH₂OCH₃, CONHCH(C₂H₅)CH₂OH, CONHCH(C₂H₅)CH₂OCH₃, CONH(CH₂)₂OCH₃, CONH(CH₂)₂OH, CONH(CH₂)₃OCH₃, CONH(CH₂)₃OH, CO₂Me, CO₂Et, CO₂Pr, CO₂iPr, CO₂Bu, CO₂secBu, CO₂isoBu, CO₂tBu, CO₂(CH₂)₂OH, CO₂(CH₂)₂OCH₃, CO₂(CH₂)₃OH, CO₂(CH₂)₃OCH₃, CH₂OH, (CH₂)₂OH, (CH₂)₃OH, (CH₂)₄OH, CH₂OMe, (CH₂)₂OMe, (CH₂)₃OMe, (CH₂)₄OMe, CH₂SH, (CH₂)₂SH, (CH₂)₃SH, (CH₂)₄SH, CH₂SMe, (CH₂)₂SMe, (CH₂)₃SMe, (CH₂)₄SMe, CH₂NH₂, CH₂NAc₂, CH₂N(COCF₃)₂, CH₂NHAc, CH₂NHCOCF₃, (CH₂)₂NH₂, (CH₂)₃NH₂, (CH₂)₄NH₂, CH₂NMe₂, (CH₂)₄NHMe, (CH₂)₂NMe₂, (CH₂)₃NHMe, (CH₂)₃NMe₂, (CH₂)₄NHMe, (CH₂)₄NMe₂, CH₂CO₂Me, (CH₂)₂CO₂Me, (CH₂)₃CO₂Me, CH₂CO₂Et, (CH₂)₂CO₂Et, (CH₂)₃CO₂Et, CH₂CO₂Pr, (CH₂)₂CO₂Pr, (CH₂)₃CO₂Pr, CH₂CO₂iPr, (CH₂)₂CO₂iPr, (CH₂)₃CO₂iPr, CH₂CO₂tBu, (CH₂)₂CO₂tBu, (CH₂)₃CO₂tBu, CH₂CO₂(CH₂)₂OH CH₂CO₂(CH₂)₂OCH₃, CH₂CO₂(CH₂)₃OH, CH₂CO₂(CH₂)₃OCH₃, CH₂NHCO₂Me, CH₂NHCO₂tBu, CH₂NHCO₂Et, CH₂NHCO₂Pr, CH₂NHCO₂iPr, CH₂NHCO₂Bu, CH₂NHCO₂tBu, CH₂NHCO₂secBu, CH₂NHCO₂isoBu, methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl; CF₃, CF₂H, CCl₃, CH₂CF₃, C₂F₅, C₃F₇, CF(CF₃)₂, SiMe₃, SiMe₂tBu, SiMe₂Ph, cyclopropyl, cyclobutyl, cyclopentyl, substituted or unsubstituted cyclohexyl, substituted or unsubstituted phenyl, substituted or unsubstituted benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methylpiperazin-1-yl, morpholin-1-yl, 1-pyrrolidin-2-one, 1-piperidin-2-one, 1-azetidin-2-one.
A very particularly preferably represents hydrogen, fluorine, chlorine, cyano, nitro, methoxy, ethoxy, benzyloxy, t-butoxy, —O(CH₂)₂OCH₃, phenyloxy, i-propanyloxy, trifluoromethoxy, trichloromethoxy, S-Me, S-Et, S-tBu, SCF₃, SO-Me, SO-Et, SO-tBu, SO₂-Me, SO₂-Et, SONHMe, SONMe₂, SONHEt, SONEt₂, SONHCF₃, SON(CF₃)₂, SO₂NHMe, SO₂NMe₂, SO₂NEt₂, SO₂NHEt, SO₂NHCF₃, SO₂N(CF₃)₂, COMe, COEt, COtBu, COCF₃, NHCO₂Me, NHCO₂Et, NHCO₂tBu, NHCOMe, NHCOEt, NHCOtBu, N(Me)COMe, N(Me)COEt, N(Me)COtBu, N(Me)CO(CH₂)₂OCH₃, NMe₂, NEt₂, NHMe, NH₂, NHtBu, NHEt, NHtBu, NHSOMe, NHSO₂Me, NHSOEt, NHSO₂Et, NMeSOMe, NMeSO₂Me, NMeSOEt, NMeSO₂Et, NHSOCF₃, NHSO₂CF₃, OCONHMe, OCONHEt, OCONHtBu, OCONMe₂, OCONEt₂, OCOMe, OCOEt, OCOtBu, CONHEt, CONEt₂, CONHMe, CONMe₂, CONHtBu, CO₂Me, CO₂Et, CO₂tBu, CO₂(CH₂)₂OCH₃, CH₂OMe, (CH₂)₂OMe, CH₂NH₂, CH₂NAc₂, CH₂N(COCF₃)₂, CH₂NHAc, CH₂NHCOCF₃, (CH₂)₂NH₂, (CH₂)₃NH₂, CH₂NMe₂, (CH₂)₂NHMe, (CH₂)₂NMe₂, CH₂CO₂Me, (CH₂)₂CO₂Me, CH₂CO₂Et, (CH₂)₂CO₂Et, CH₂CO₂tBu, (CH₂)₂CO₂tBu, CH₂NHCO₂Me, CH₂NHCO₂tBu, CH₂NHCO₂Et, CH₂NHCO₂tBu, methyl, ethyl, propyl, 1-methylethyl, butyl, 1-methylpropyl, 2-methylpropyl, 1,1-dimethylethyl, CF₃, CF₂H, CCl₃, CH₂CF₃, SiMe₃, SiMe₂tBu, cyclopropyl, cyclobutyl, cyclopentyl, substituted or unsubstituted cyclohexyl, substituted or unsubstituted phenyl, substituted or unsubstituted benzyl, pyridin-4-yl, pyridin-3-yl, pyridin-2-yl, thien-2-yl, thien-3-yl, furan-2-yl, furan-3-yl, pyrrol-2-yl, pyrrol-3-yl, piperidin-1-yl, piperazin-1-yl, 4-methyl piperazin-1-yl, morpholin-1-yl, 1-pyrrolidin-2-one, 1-piperidin-2-one, 1-azetidin-2-one.

Two adjacent radicals together with the carbon atoms to which they are attached preferably form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent. Two adjacent radicals together with the carbon atoms to which they are attached particularly preferably form an unsubstituted or substituted, unsaturated or fully or partially saturated heterocyclic or carbocyclic 5- to 7-membered ring which, depending on the ring size, may contain up to 2 further heteroatoms selected from the group consisting of N, O and S, where two oxygen atoms are not adjacent.
R⁹ and R¹⁰ independently of one another preferably represent hydrogen, C₁-C₆-alkyl, vinyl, allyl, ethynyl, propargyl, C₁-C₆-haloalkyl having in each case 1 to 13 fluorine, chlorine and/or bromine atoms, C₃-C₆-cycloalkyl, hydroxy-C₁-C₄-alkyl, C₁-C₄-alkoxy-C₁-C₄-alkyl, (C₁-C₄-alkyl)thio(C₁-C₄-alkyl), phenyl, benzyl, pyridinyl, pyridinylmethyl, represent phenyl or benzyl, each of which is mono- or polysubstituted by identical or different substituents from the group consisting of fluorine, chlorine, bromine, C₁-C₄-alkyl, C₁-C₄-alkoxy, C₁-C₄-haloalkyl having in each case 1 to 9 fluorine, chlorine and/or bromine atoms.
Moreover, in the case that two radicals R⁹ and R¹⁰ are attached to a nitrogen atom, these two radicals together preferably with the nitrogen atom to which they are attached form a 3- to 6-membered unsubstituted or substituted, saturated or unsaturated cycle which, depending on the ring size, may contain up to 2 further heteroatoms selected from the group consisting of N, O and S, where two oxygen atoms are not adjacent.
Moreover, the two radicals R⁹ and R¹⁰ in the grouping NR⁹COR¹⁰ together with this grouping to which they are attached preferably form a 4- to 6-membered unsubstituted or substituted, saturated or unsaturated cycle which, depending on the ring size, may contain up to 2 further heteroatoms selected from the group consisting of N, O and S, where two oxygen atoms are not adjacent
   m preferably represents the number 1, 2, 3, 4 or 5.
   m particularly preferably represents the number 1, 2, 3 or 4.

However, the abovementioned general or preferred radical definitions or illustrations can be combined with one another as desired, i.e. including combinations between the respective ranges and preferred ranges. They apply both to the end products and, correspondingly, to precursors and intermediates. Moreover, individual definitions may not apply.

Preference is given to compounds of the formula (I) in which all radicals in each case have the preferred meanings mentioned above.

Particular preference is given to compounds of the formula (I) in which all radicals in each case have the particularly preferred meanings mentioned above.

Very particular preference is given to compounds of the formula (I) in which all radicals in each case have the very particularly preferred meanings mentioned above.

Special preference is furthermore given to compounds of the formula (I) in which R¹ represents halogen, nitro, CF₃, OCF₃, SCF₃, C₁-C₄-alkyl, C₁-C₄-alkoxy or benzyloxy, where the other substituents have one or more of the preferred meanings mentioned above, and also to agrochemically active salts thereof.

Special preference is furthermore given to compounds of the formula (I) in which R¹ represents bromine, chlorine, fluorine, iodine, nitro, CF₃, OCF₃, SCF₃, methyl, methoxy, ethoxy or benzyloxy, where the other substituents have one or more of the meanings mentioned above, and also to agrochemically active salts thereof.

Special preference is furthermore given to compounds of the formula (I) in which R² represents halogen, nitro, CF₃, C₁-C₄-alkyl or C₁-C₄-alkoxy, where the other substituents have one or more of the meanings mentioned above, and also to agrochemically active salts thereof.

Special preference is furthermore given to compounds of the formula (I) in which R² represents chlorine, fluorine, nitro, CF₃, methyl or methoxy, where the other substituents have one or more of the meanings mentioned above, and also to agrochemically active salts thereof.

Special preference is furthermore given to compounds of the formula (I) in which R³ represents halogen, nitro, CF₃, OCF₃, C₁-C₄-alkoxy or benzyloxy, where the other substituents have one or more of the meanings mentioned above, and also to agrochemically active salts thereof.

Special preference is furthermore given to compounds of the formula (I) in which R³ represents chlorine, fluorine, nitro, CF₃, OCF₃, methyl, t-butyl, methoxy, ethoxy, propan-2-yloxy or benzyloxy, where the other substituents have one or more of the meanings mentioned above, and also to agrochemically active salts thereof.

Special preference is furthermore given to compounds of the formula (I) in which
R¹ represents halogen, nitro, CF₃, OCF₃, SCF₃, C₁-C₄-alkyl, C₁-C₄-alkoxy or benzyloxy,
R² represents H, halogen, C₁-C₄-alkyl or C₁-C₄-alkoxy,
R³ represents H, halogen, C₁-C₄-alkyl or C₁-C₄-alkoxy,
R⁴ represents H, halogen or C₁-C₄-alkyl,
R⁵ represents H, halogen or C₁-C₄-alkoxy,
R⁶, R⁷ and R⁸ independently of one another represent H, chlorine, bromine, methyl, ethyl, phenyl, methoxymethyl, methylsulphanyl or nitro,
   and the agrochemically active salts thereof.

Special preference is furthermore given to compounds of the formula (I) in which
R¹ represents bromine, chlorine, fluorine, iodine, nitro, CF₃, OCF₃, SCF₃, methyl, methoxy, ethoxy or benzyloxy,
R² represents H, chlorine, methyl or methoxy,
R³ represents H, chlorine, methyl or methoxy,
R⁴ represents H, bromine, fluorine, chlorine or methyl,
R⁵ represents H, methoxy or fluorine,
R⁶, R⁷ and R⁸ independently of one another represent H, chlorine, bromine, methyl, ethyl, phenyl, methoxymethyl, methylsulphanyl or nitro,
   and the agrochemically active salts thereof.

Special preference is furthermore given to compounds of the formula (I) in which
R¹ represents H,
R² represents halogen, nitro, CF₃, C₁-C₄-alkyl or C₁-C₄-alkoxy,
R³ represents H, halogen, C₁-C₄-alkyl or C₁-C₄-alkoxy,
R⁴ represents H, halogen, C₁-C₄-alkyl or C₁-C₄-alkoxy,
R⁵ represents H,
R⁶, R⁷ and R⁸ independently of one another represent H, chlorine, bromine, methyl, ethyl, phenyl, methoxymethyl, methylsulphanyl or nitro,
   and the agrochemically active salts thereof.

Special preference is furthermore given to compounds of the formula (I) in which
R¹ represents H,
R² represents fluorine, chlorine, nitro, CF₃, methyl or methoxy,
R³ represents H, chlorine, methyl or methoxy,
R⁴ represents H, chlorine, methoxy or methyl,
R⁵ represents H,
R⁶, R⁷ and R⁸ independently of one another represent H, chlorine, bromine, methyl, ethyl, phenyl, methoxymethyl, methylsulphanyl or nitro,
   and the agrochemically active salts thereof.

Special preference is furthermore given to compounds of the formula (I) in which
R¹ represents H,
R² represents H,
R³ represents halogen, nitro, CF₃, OCF₃, C₁-C₄-alkyl, C₁-C₄-alkoxy or benzyloxy,
R⁴ represents H,
R⁵ represents H,
R⁶, R⁷ and R⁸ independently of one another represent H, chlorine, bromine, methyl, ethyl, phenyl, methoxymethyl, methylsulphanyl or nitro,
   and the agrochemically active salts thereof.

Special preference is furthermore given to compounds of the formula (I) in which
R¹ represents H,
R² represents H,
R³ represents chlorine, fluorine, nitro, CF₃, OCF₃, methyl, t-butyl, methoxy, ethoxy, propan-2-yloxy or benzyloxy,
R⁴ represents H,
R⁵ represents H,
R⁶, R⁷ and R⁸ independently of one another represent H, chlorine, bromine, methyl, ethyl, phenyl, methoxymethyl, methylsulphanyl or nitro,
   and the agrochemically active salts thereof.

The compounds of the formula (I) may be present either in pure form or as mixtures of different possible isomeric forms, especially of stereoisomers, such as E and Z, threo and erythro, and also optical isomers, such as R and S isomers or atropisomers, and, if appropriate, also of tautomers. What is claimed are both the E and the Z isomers, and also the threo and erythro, and also the optical isomers, any mixtures of these isomers, and also the possible tautomeric forms.

Depending on the nature of the substituents defined above, the compounds of the formula (I) have acidic or basic properties and can form salts, if appropriate also inner salts, or adducts with inorganic or organic acids or with bases or with metal ions.

Suitable metal ions are in particular the ions of the elements of the second main group, in particular calcium and magnesium, of the third and fourth main group, in particular aluminium, tin and lead, and also of the first to eighth transition group, in particular chromium, manganese, iron, cobalt, nickel, copper, zinc and others.

Particular preference is given to the metal ions of the elements of the fourth period. Here, the metals can be present in the various valencies that they can assume.

If the compounds of the formula (I) carry hydroxyl, carboxyl or other groups which induce acidic properties, these compounds can be reacted with bases to give salts. Suitable bases are, for example, hydroxides, carbonates, hydrogencarbonates of the alkali metals and alkaline earth metals, especially those of sodium, potassium, magnesium and calcium, and also ammonia, primary, secondary and tertiary amines having C₁-C₄-alkyl radicals, mono-, di- and trialkanolamines of C₁-C₄-alkanols, choline and chlorocholine.

If the compounds of the formula (I) carry amino, alkylamino or other groups which induce basic properties, these compounds can be reacted with acids to give salts, or they are directly obtained as salts in the synthesis. Examples of inorganic acids are hydrohalic acids, such as hydrogen fluoride, hydrogen chloride, hydrogen bromide and hydrogen iodide, sulphuric acid, phosphoric acid and nitric acid, and acidic salts, such as Na—HSO₄ and KHSO₄.

Suitable organic acids are, for example, formic acid, carbonic acid and alkanoic acids, such as acetic acid, trifluoroacetic acid, trichloroacetic acid and propionic acid, and also glycolic acid, thiocyanic acid, lactic acid, succinic acid, citric acid, benzoic acid, cinnamic acid, oxalic acid, alkylsulphonic acids (sulphonic acids having straight-chain or branched alkyl radicals of 1 to 20 carbon atoms), arylsulphonic acids or aryldisulphonic acids (aromatic radicals, such as phenyl and naphthyl, which carry one or two sulphonic acid groups), alkylphosphonic acids (phosphonic acids having straight-chain or branched alkyl radicals of 1 to 20 carbon atoms), arylphosphonic acids or aryldiphosphonic acids (aromatic radicals, such as phenyl and naphthyl, which carry one or two phosphonic acid radicals), where the alkyl and aryl radicals may carry further substituents, for example p-toluenesulphonic acid, salicylic acid, p-aminosalicylic acid, 2-phenoxybenzoic acid, 2-acetoxybenzoic acid, etc. The salts obtainable in this manner also have fungicidal properties.

In the definitions of the symbols given in the formulae above, collective terms were used which are generally representative of the following substituents:

halogen: fluorine, chlorine, bromine and iodine;
alkyl: saturated, straight-chain or branched hydrocarbon radicals having 2 to 8 carbon atoms, for example C₁-C₆-alkyl, such as methyl, ethyl, propyl, 1-methylethyl, butyl, 1-methylpropyl, 2-methylpropyl, 1,1-dimethylethyl, pentyl, 1-methylbutyl, 2-methylbutyl, 3-methylbutyl, 2,2-dimethylpropyl, 1-ethylpropyl, hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl and 1-ethyl-2-methylpropyl;
haloalkyl: straight-chain or branched alkyl groups having 1 to 8 carbon atoms (as mentioned above), where in these groups some or all of the hydrogen atoms may be replaced by halogen atoms as mentioned above, for example C₁-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl and 1,1,1-trifluoroprop-2-yl;
alkenyl: unsaturated, straight-chain or branched hydrocarbyl radicals having 2 to 8 carbon atoms and one double bond in any position, for example C₂-C₆-alkenyl, such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl;
alkynyl: straight-chain or branched hydrocarbyl groups having 2 to 8 carbon atoms and one triple bond in any position, for example C₂-C₆-alkynyl, such as ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl;
alkoxy: saturated, straight-chain or branched alkoxy radicals having 1 to 8 carbon atoms, for example (but not limited thereto) C₁-C₆-alkoxy, such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 2,2-dimethylpropoxy, 1-ethylpropoxy, hexoxy, 1,1-dimethylpropoxy, 1,2-dimethylpropoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy;
alkylthio: saturated, straight-chain or branched alkylthio radicals having 1 to 8 carbon atoms, for example (but not limited thereto) C₁-C₆-alkylthio, such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio;
alkylsulphinyl: saturated, straight-chain or branched alkylsulphinyl radicals having 1 to 8 carbon atoms, for example (but not limited to) C₁-C₆-alkylsulphinyl, such as methylsulphinyl, ethylsulphinyl, propylsulphinyl, 1-methylethylsulphinyl, butylsulphinyl, 1-methylpropylsulphinyl, 2-methylpropylsulphinyl, 1,1-dimethylethylsulphinyl, pentylsulphinyl, 1-methylbutylsulphinyl, 2-methylbutylsulphinyl, 3-methylbutylsulphinyl, 2,2-dimethylpropylsulphinyl, 1-ethylpropylsulphinyl, hexylsulphinyl, 1,1-dimethylpropylsulphinyl, 1,2-dimethylpropylsulphinyl, 1-methylpentylsulphinyl, 2-methylpentylsulphinyl, 3-methylpentylsulphinyl, 4-methylpentylsulphinyl, 1,1-dimethylbutylsulphinyl, 1,2-dimethylbutylsulphinyl, 1,3-dimethylbutylsulphinyl, 2,2-dimethylbutylsulphinyl, 2,3-dimethylbutylsulphinyl, 3,3-dimethylbutylsulphinyl, 1-ethylbutylsulphinyl, 2-ethylbutylsulphinyl, 1,1,2-trimethylpropylsulphinyl, 1,2,2-trimethylpropylsulphinyl, 1-ethyl-1-methylpropylsulphinyl and 1-ethyl-2-methylpropylsulphinyl;
alkylsulphonyl: saturated, straight-chain or branched alkylsulphonyl radicals having 1 to 8 carbon atoms, for example (but not limited thereto) C₁-C₆-alkylsulphonyl, such as methylsulphonyl, ethylsulphonyl, propylsulphonyl, 1-methylethylsulphonyl, butylsulphonyl, 1-methylpropylsulphonyl, 2-methylpropylsulphonyl, 1,1-dimethylethylsulphonyl, pentylsulphonyl, 1-methylbutylsulphonyl, 2-methylbutylsulphonyl, 3-methylbutylsulphonyl, 2,2-dimethylpropylsulphonyl, 1-ethylpropylsulphonyl, hexylsulphonyl, 1,1-dimethylpropylsulphonyl, 1,2-dimethylpropylsulphonyl, 1-methylpentylsulphonyl, 2-methylpentylsulphonyl, 3-methylpentylsulphonyl, 4-methylpentylsulphonyl, 1,1-dimethylbutylsulphonyl, 1,2-dimethylbutylsulphonyl, 1,3-dimethylbutylsulphonyl, 2,2-dimethylbutylsulphonyl, 2,3-dimethylbutylsulphonyl, 3,3-dimethylbutylsulphonyl, 1-ethylbutylsulphonyl, 2-ethylbutylsulphonyl, 1,1,2-trimethylpropylsulphonyl, 1,2,2-trimethylpropylsulphonyl, 1-ethyl-1-methylpropylsulphonyl and 1-ethyl-2-methylpropylsulphonyl;
cycloalkyl: monocyclic saturated hydrocarbon groups having 3 to 8 carbon ring members, such as cyclopropyl, cyclobutyl, cyclopentyl and cyclohexyl;
cycloalkenyl: monocyclic non-aromatic hydrocarbon groups having 3 to 8 carbon ring members and at least one double bond, such as cyclopenten-1-yl, cyclohexen-1-yl, cyclohepta-1,3-dien-1-yl;
haloalkyl: straight-chain or branched alkyl groups having 1 to 8 carbon atoms (as mentioned above), where in these groups some or all of the hydrogen atoms may be replaced by halogen atoms as mentioned above, for example (but not limited thereto) C₃-C₃-haloalkyl, such as chloromethyl, bromomethyl, dichloromethyl, trichloromethyl, fluoromethyl, difluoromethyl, trifluoromethyl, chlorofluoromethyl, dichlorofluoromethyl, chlorodifluoromethyl, 1-chloroethyl, 1-bromoethyl, 1-fluoroethyl, 2-fluoroethyl, 2,2-difluoroethyl, 2,2,2-trifluoroethyl, 2-chloro-2-fluoroethyl, 2-chloro-2-difluoroethyl, 2,2-dichloro-2-fluoroethyl, 2,2,2-trichloroethyl, pentafluoroethyl and 1,1,1-trifluoroprop-2-yl;
heterocyclyl: a five- to ten-membered saturated or partially unsaturated heterocycle which contains one to four heteroatoms from the group consisting of oxygen, nitrogen and sulphur: mono- or bicyclic heterocycles (heterocyclyl) which contain, in addition to carbon ring members, one to three nitrogen atoms and/or one oxygen or sulphur atom or one or two oxygen and/or sulphur atoms; if the ring contains a plurality of oxygen atoms, these are not directly adjacent; for example oxiranyl, aziridinyl, 2-tetrahydrofuranyl, 3-tetrahydrofuranyl, 2-tetrahydrothienyl, 3-tetrahydrothienyl, 2-pyrrolidinyl, 3-pyrrolidinyl, 3-isoxazolidinyl, 4-isoxazolidinyl, 5-isoxazolidinyl, 3-isothiazolidinyl, 4-isothiazolidinyl, 5-isothiazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 2-imidazolidinyl, 4-imidazolidinyl, 1,2,4-oxadiazolidin-3-yl, 1,2,4-oxadiazolidin-5-yl, 1,2,4-thiadiazolidin-3-yl, 1,2,4-thiadiazolidin-5-yl, 1,2,4-triazolidin-3-yl, 1,3,4-oxadiazolidin-2-yl, 1,3,4-thiadiazolidin-2-yl, 1,3,4-triazolidin-2-yl, 2,3-dihydrofur-2-yl, 2,3-dihydrofur-3-yl, 2,4-dihydrofur-2-yl, 2,4-dihydrofur-3-yl, 2,3-dihydrothien-2-yl, 2,3-dihydrothien-3-yl, 2,4-dihydrothien-2-yl, 2,4-dihydrothien-3-yl, 2-pyrrolin-2-yl, 2-pyrrolin-3-yl, 3-pyrrolin-2-yl, 3-pyrrolin-3-yl, 2-isoxazolin-3-yl, 3-isoxazolin-3-yl, 4-isoxazolin-3-yl, 2-isoxazolin-4-yl, 3-isoxazolin-4-yl, 4-isoxazolin-4-yl, 2-isoxazolin-5-yl, 3-isoxazolin-5-yl, 4-isoxazolin-5-yl, 2-isothiazolin-3-yl, 3-isothiazolin-3-yl, 4-isothiazolin-3-yl, 3-isothiazolin-4-yl, 4-isothiazolin-4-yl, 2-isothiazolin-5-yl, 3-isothiazolin-5-yl, 4-isothiazolin-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 3,4-dihydrooxazol-2-yl, 3,4-dihydrooxazol-3-yl, 3,4-dihydrooxazol-4-yl, 2-piperidinyl, 3-piperidinyl, 4-piperidinyl, 1,3-dioxan-5-yl, 2-tetrahydropyranyl, 4-tetrahydropyranyl, 2-tetrahydrothienyl, 3-hexahydropyridazinyl, 4-hexahydropyridazinyl, 2-hexahydropyrimidinyl, 4-hexahydropyrimidinyl, 5-hexahydropyrimidinyl, 2-piperazinyl, 1,3,5-hexahydrotriazin-2-yl and 1,2,4-hexahydrotriazin-3-yl;
heteroaryl: a 5 or 6-membered fully unsaturated monocyclic ring system comprising one to four heteroatoms from the group consisting of oxygen, nitrogen and sulphur; if the ring contains a plurality of oxygen atoms, these are not directly adjacent;
5-membered heteroaryl which contains one to four nitrogen atoms or one to three nitrogen atoms and one sulphur or oxygen atom: 5-membered heteroaryl groups which, in addition to carbon atoms, may contain one to four nitrogen atoms or one to three nitrogen atoms and one sulphur or oxygen atom as ring members, for example (but not limited thereto) 2-furyl, 3-furyl, 2-thienyl, 3-thienyl, 2-pyrrolyl, 3-pyrrolyl, 3-isoxazolyl, 4-isoxazolyl, 5-isoxazolyl, 3-isothiazolyl, 4-isothiazolyl, 5-isothiazolyl, 3-pyrazolyl, 4-pyrazolyl, 5-pyrazolyl, 2-oxazolyl, 4-oxazolyl, 5-oxazolyl, 2-thiazolyl, 4-thiazolyl, 5-thiazolyl, 2-imidazolyl, 4-imidazolyl, 1,2,4-oxadiazol-3-yl, 1,2,4-oxadiazol-5-yl, 1,2,4-thiadiazol-3-yl, 1,2,4-thiadiazol-5-yl, 1,2,4-triazol-3-yl, 1,3,4-oxadiazol-2-yl, 1,3,4-thiadiazol-2-yl and 1,3,4-triazol-2-yl;
5-membered heteroaryl which contains one to four nitrogen atoms and is attached via nitrogen or benzofused 5-membered heteroaryl which contains one to three nitrogen atoms and is attached via nitrogen: 5-membered heteroaryl groups which, in addition to carbon atoms, may contain one to four nitrogen atoms or one to three nitrogen atoms as ring members and in which two adjacent carbon ring members or one nitrogen and one adjacent carbon ring member may be bridged by a buta-1,3-diene-1,4-diyl group in which one or two carbon atoms may be replaced by nitrogen atoms, where these rings are attached to the skeleton via one of the nitrogen ring members, for example (but not limited thereto) 1-pyrrolyl, 1-pyrazolyl, 1,2,4-triazol-1-yl, 1-imidazolyl, 1,2,3-triazol-1-yl, 1,3,4-triazol-1-yl;
6-membered heteroaryl which contains one to four nitrogen atoms: 6-membered heteroaryl groups which, in addition to carbon atoms, may contain, respectively, one to three and one to four nitrogen atoms as ring members, for example (but not limited thereto) 2-pyridinyl, 3-pyridinyl, 4-pyridinyl, 3-pyridazinyl, 4-pyridazinyl, 2-pyrimidinyl, 4-pyrimidinyl, 5-pyrimidinyl, 2-pyrazinyl, 1,3,5-triazin-2-yl, 1,2,4-triazin-3-yl and 1,2,4,5-tetrazin-3-yl;
benzofused 5-membered heteroaryl containing one to three nitrogen atoms or one nitrogen atom and one oxygen or sulphur atom: for example (but not limited to) indol-1-yl, indol-2-yl, indol-3-yl, indol-4-yl, indol-5-yl, indol-6-yl, indol-7-yl; benzimidazol-1-yl, benzimidazol-2-yl, benzimidazol-4-yl, benzimidazol-5-yl, indazol-1-yl, indazol-3-yl, indazol-4-yl, indazol-5-yl, indazol-6-yl, indazol-7-yl, indazol-2-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,3-benzoxazol-2-yl, 1,3-benzoxazol-4-yl, 1,3-benzoxazol-5-yl, 1,3-benzoxazol-6-yl and 1,3-benzoxazol-7-yl;
benzo-fused 6-membered heteroaryl which contains one to three nitrogen atoms: for example (but not limited thereto) quinolin-2-yl, quinolin-3-yl, quinolin-4-yl, quinolin-5-yl, quinolin-6-yl, quinolin-7-yl, quinolin-8-yl, isoquinolin-1-yl, isoquinolin-3-yl, isoquinolin-4-yl, isoquinolin-5-yl, isoquinolin-6-yl, isoquinolin-7-yl and isoquinolin-8-yl.

Furthermore, it has been found that 3-aryl-4-(2-thienylmethylene)isoxazol-5(4H)-ones of the formula (I) are obtained according to the scheme below:

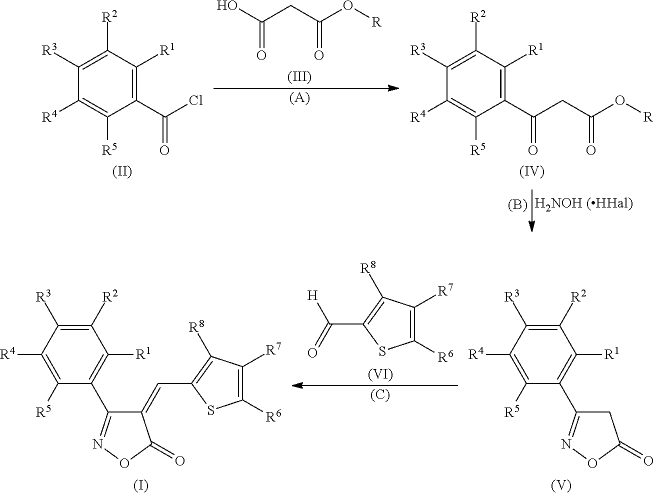

3-aryl-4-(2-thienylmethylene)isoxazol-5(4H)-ones of the formula (I) are obtained when 3-arylisoxazol-5(4H)-ones of the formula (V) are, if appropriate in the presence of a base and a diluent, reacted with thiophene-2-carbaldehydes of the formula (VI) [cf. the scheme above, step (C)]. In these formulae, R¹ to R⁸ preferably, particularly preferable and very particularly preferably represent those radicals which have already been defined in connection with the description of the compounds of the formula (I) according to the invention as being preferred, particularly preferred and very particularly preferred etc.

3-Arylisoxazol-5(4H)-ones of the formula (V) can be obtained by condensation of β-keto esters of the formula (IV) with hydroxylamine, if appropriate in the presence of a diluent. Alternatively, it is also possible to use hydroxylamine halogen salts in the presence of a base [cf. the scheme above, step (B)].

β-Keto esters of the formula (IV) can likewise be obtained by literature methods, for example by reacting benzoyl chlorides of the formula (II) with monoalkyl malonates of the formula (III) in which R represents alkyl, preferably methyl or ethyl, in the presence of a base or by reacting with metal salts of monoalkyl malonates of the formula (III), preferably alkali metal or alkaline earth metal salts such as, for example, sodium, potassium, calcium or magnesium salts, in each case optionally in the presence of a Lewis acid and a diluent [cf. the scheme above, step (A)].

The benzoyl chlorides of the formula (II) and the monoalkyl malonates of the formula (III) are either commercially available or can be obtained by literature procedures from commercially available precursors.

Suitable bases for carrying out steps (A) to (C) are, if appropriate, the customary inorganic or organic bases or acid binders. These preferably include ammonium, alkali metal or alkaline earth metal acetates, amides, carbonates, bicarbonates, hydrides, hydroxides or alkoxides, such as, for example, sodium acetate, potassium acetate or calcium acetate, lithium amide, sodium amide, potassium amide or calcium amide, sodium carbonate, potassium carbonate, calcium carbonate or caesium carbonate, sodium bicarbonate, potassium bicarbonate or calcium bicarbonate, lithium hydride, sodium hydride, potassium hydride or calcium hydride, lithium hydroxide, sodium hydroxide, potassium hydroxide or calcium hydroxide, sodium methoxide, ethoxide, n- or i-propoxide, n-, i-, s- or t-butoxide or potassium methoxide, ethoxide, n- or i-propoxide, n-, s- or t-butoxide; furthermore also basic organic nitrogen compounds, such as, for example, trimethylamine, triethylamine, tripropylamine, tributylamine, ethyldiisopropylamine, N,N-dimethylcyclohexylamine, dicyclohexylamine, ethyldicyclohexylamine, N,N-dimethylaniline, N,N-dimethylbenzylamine, pyridine, 2-methyl-, 3-methyl-, 4-methyl-, 2,4-dimethyl-, 2,6-dimethyl-, 3,4-dimethyl- and 3,5-dimethylpyridine, 5-ethyl-2-methylpyridine, 4-dimethylaminopyridine, N-methylpiperidine, morpholine, 1,4-diazabicyclo[2.2.2]octane (DABCO), 1,5-diazabicyclo[4.3.0]non-5-ene (DBN), 1,8-diazabicyclo[5.4.0]undec-7-ene (DBU), or phosphazene bases such as, for example, P[N═P(NMe₂)₃]=NtBu. Their amounts may vary between catalytic, stoichiometric and excess amounts. When preparing the compounds described in the present patent, in step (A) it is preferred to use excess triethylamine, in step (B), if hydroxylamine hydrochloride is used, it is preferred to employ excess sodium bicarbonate and in step (C) it is preferred to use morpholine or ammonium acetate in catalytic or stoichiometric amounts.

Suitable Lewis acids for carrying out step (A) are, if appropriate, the customary halides of the metals of the 2nd and 3rd main group and transition group of the Periodic System, such as zinc chloride, boron trifluoride, aluminium trichloride or magnesium chloride. When preparing the compounds described in the present patent, preference is given to using excess magnesium chloride.

Steps (A) to (C) in the preparation of the compounds according to the invention are preferably carried out using one or more diluents. Suitable diluents for step (A) are virtually all aprotic organic solvents. These include, in particular, aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, chloroform, carbon tetrachloride, chlorobenzene and o-dichlorobenzene, ethers such as diethyl ether and dibutyl ether, glycol dimethyl ether and diglycol dimethyl ether, tetrahydrofuran and dioxane, ketones, such as acetone, methyl ethyl ketone, methyl isopropyl ketone and methyl isobutyl ketone, esters, such as methyl acetate and ethyl acetate, nitriles such as, for example, acetonitrile and propionitrile, amides such as, for example, dimethylformamide, dimethylacetamide and N-methylpyrrolidone, and also dimethyl sulphoxide, tetramethylene sulphone and hexamethylphosphortriamide. When preparing the compounds described in the present patent, preference is given to using acetonitrile or tetrahydrofuran in step (A).

Suitable diluents for step (B) are primarily polar protic organic solvents, in particular alcohols such as, for example, methanol, ethanol, propanol, isopropanol, butanol, isobutanol, 2-methoxyethanol. Preference is given to using ethanol and methanol or mixtures thereof with dichloromethane.

Preferred diluents for step (C) are aliphatic and aromatic, optionally halogenated hydrocarbons, such as pentane, hexane, heptane, cyclohexane, petroleum ether, benzine, ligroin, benzene, toluene, xylene, methylene chloride, ethylene chloride, dichloromethane, chloroform, carbon tetrachloride, chlorobenzene and o-dichlorobenzene, ethers such as diethyl ether and dibutyl ether, glycol dimethyl ether and diglycol dimethyl ether, tetrahydrofuran and dioxane, ketones, such as acetone, methyl ethyl ketone, methyl isopropyl ketone and methyl isobutyl ketone, esters, such as methyl acetate and ethyl acetate, nitriles such as, for example, acetonitrile and propionitrile, amides such as, for example, dimethylformamide, dimethylacetamide and N-methylpyrrolidone, and also dimethyl sulphoxide, tetramethylene sulphone and hexamethylphosphoric triamide. When preparing the compounds described in the present patent, preference is given to using dichloromethane, chloroform and toluene in step (C).

The reaction temperatures in steps (A) to (C) in the preparation of the compounds according to the invention can be varied within a relatively wide range. In general, depending on the chosen diluent, step (A) is carried out with cooling at temperatures between −20° C. and 40° C., steps (B) and (C) are carried out at room temperature or with warming at temperatures between 25° C. and 100° C., but always at temperatures which do not exceed the boiling point of the solvent used.

The reaction steps (A) to (C) are generally carried out at atmospheric pressure.

When carrying out step (A), usually 1 mol of benzoyl chloride of the formula (II) is reacted with an excess, preferably 2 mol, of monoalkyl malonate salts of the formula (III) (preferably potassium or lithium ethyl or methyl malonate) in the presence of an excess, preferably 2-3 mol, of base and an excess, preferably 2 mol, of catalyst. However, it is also possible to employ the reaction components in other ratios. Workup is effected by customary methods. In general, water is added and the reaction mixture is neutralized with an acid, preferably hydrochloric acid, and the organic phase is then separated off and, after drying, concentrated under reduced pressure. The residue that remains can, if appropriate, be freed from any impurities that may still be present using customary methods such as chromatography or recrystallization.

When carrying out step (B), usually 1 mol of β-keto ester of the formula (IV) is reacted with an excess, preferably 3 mol, of hydroxylamine or hydroxylamine hydrochloride and also an excess, preferably 3 mol, of a base, preferably sodium bicarbonate. However, it is also possible to employ the reaction components in other ratios. Workup is effected by customary methods. In general, the reaction mixture is concentrated under reduced pressure, water is added and the reaction mixture is acidified with an acid, preferably dilute hydrochloric acid, and the solid which precipitates is filtered off with suction. The product isolated in this manner can, if appropriate, be freed from any impurities that may still be present using customary methods such as chromatography or recrystallization.

When carrying out step (C), usually 1 mol of 3-arylisoxazol-5(4H)-one of the formula (V) is reacted with a slight excess of from 1.05 to 1.2 mol of thiophene-2-carbaldehyde of the formula (VI) and also varying amounts of base (catalytic to stoichiometric). However, it is also possible to employ the reaction components in other ratios. Workup is effected by customary methods. In general, the reaction mixture is concentrated under reduced pressure and, if required resuspended in ethanol and filtered off with suction, and the resulting solid is then freed from any impurities that may still be present using customary methods such as chromatography or recrystallization.

The present invention furthermore relates to a crop protection composition for controlling unwanted fungi, which composition comprises at least one of the compounds of the formula (I). These are preferably fungicidal compositions which comprise agriculturally suitable auxiliaries, solvents, carriers, surfactants or extenders.

Moreover, the invention relates to a method for controlling unwanted microorganisms characterized in that, according to the invention, compounds of the formula (I) are applied to the phytopathogenic fungi and/or their habitat.

According to the invention, a carrier is a natural or synthetic organic or inorganic substance with which the active compounds are mixed or bonded for better applicability, in particular for application to plants or plant parts or seed. The carrier, which may be solid or liquid, is generally inert and should be suitable for use in agriculture.

Suitable solid or liquid carriers are: for example ammonium salts and ground natural minerals, such as kaolins, clays, talc, chalk, quartz, attapulgite, montmorillonite or diatomaceous earth, and ground synthetic minerals, such as finely divided silica, alumina and natural or synthetic silicates, resins, waxes, solid fertilizers, water, alcohols, especially butanol, organic solvents, mineral and vegetable oils and derivatives of these. Mixtures of such carriers may also be used. Useful solid carriers for granules include: for example crushed and fractionated natural rocks such as calcite, marble, pumice, sepiolite, dolomite, and synthetic granules of inorganic and organic meals, and also granules of organic material such as sawdust, coconut shells, maize cobs and tobacco stalks.

Suitable liquefied gaseous extenders or carriers are liquids which are gaseous at ambient temperature and under atmospheric pressure, for example aerosol propellants, such as halogenated hydrocarbons, and also butane, propane, nitrogen and carbon dioxide.

Tackifiers such as carboxymethylcellulose and natural and synthetic polymers in the form of powders, granules or latices, such as gum arabic, polyvinyl alcohol and polyvinyl acetate, or else natural phospholipids such as cephalins and lecithins and synthetic phospholipids can be used in the formulations. Further additives may be mineral and vegetable oils.

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or dichloromethane, aliphatic hydrocarbons such as cyclohexane or paraffins, for example mineral oil fractions, mineral and vegetable oils, alcohols such as butanol or glycol and their ethers and esters, ketones such as acetone, methyl ethyl ketone, methyl isobutyl ketone or cyclohexanone, strongly polar solvents such as dimethylformamide and dimethyl sulphoxide, and also water.

The compositions according to the invention may comprise additional further components, such as, for example, surfactants. Suitable surfactants are emulsifiers and/or foam formers, dispersants or wetting agents having ionic or nonionic properties, or mixtures of these surfactants. Examples of these are salts of polyacrylic acid, salts of lignosulphonic acid, salts of phenolsulphonic acid or naphthalenesulphonic acid, polycondensates of ethylene oxide with fatty alcohols or with fatty acids or with fatty amines, substituted phenols (preferably alkylphenols or arylphenols), salts of sulphosuccinic esters, taurine derivatives (preferably alkyl taurates), phosphoric esters of polyethoxylated alcohols or phenols, fatty esters of polyols, and derivatives of the compounds containing sulphates, sulphonates and phosphates, for example alkylaryl polyglycol ethers, alkylsulphonates, alkyl sulphates, arylsulphonates, protein hydrolysates, lignosulphite waste liquors and methylcellulose. The presence of a surfactant is required if one of the active compounds and/or one of the inert carriers is insoluble in water and when the application takes place in water. The proportion of surfactants is between 5 and 40 percent by weight of the composition according to the invention.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

Stabilizers, such as low-temperature stabilizers, preservatives, antioxidants, light stabilizers or other agents which improve chemical and/or physical stability, may also be present.

If appropriate, other additional components may also be present, for example protective colloids, binders, adhesives, thickeners, thixotropic substances, penetrants, stabilizers, sequestering agents, complex formers. In general, the active compounds can be combined with any solid or liquid additive customarily used for formulation purposes.

The formulations generally comprise between 0.05 and 99% by weight, 0.01 and 98% by weight, preferably between 0.1 and 95% by weight, particularly preferably between 0.5 and 90% of active compound, very particularly preferably between 10 and 70% by weight. The active compound content of the use forms prepared from the commercially available formulations can vary within wide limits. The active ingredient concentration of the application forms may be from 0.00000001 to 95% by weight of active compound, preferably between 0.00001 and 1% by weight. The compounds are employed in a customary mariner appropriate for the use forms.

The active compounds or compositions according to the invention can be used as such or, depending on their respective physical and/or chemical properties, in the form of their formulations or the use forms prepared therefrom, such as aerosols, capsule suspensions, cold-fogging concentrates, warm-fogging concentrates, encapsulated granules, fine granules, flowable concentrates for the treatment of seed, ready-to-use solutions, dustable powders, emulsifiable concentrates, oil-in-water emulsions, water-in-oil emulsions, macrogranules, microgranules, oil-dispersible powders, oil-miscible flowable concentrates, oil-miscible liquids, foams, pastes, pesticide coated seed, suspension concentrates, suspoemulsion concentrates, soluble concentrates, suspensions, wettable powders, soluble powders, dusts and granules, water-soluble granules or tablets, water-soluble powders for the treatment of seed, wettable powders, natural products and synthetic substances impregnated with active compound, and also microencapsulations in polymeric substances and in coating materials for seed, and also ULV cold-fogging and warm-fogging formulations.

The formulations mentioned can be prepared in a manner known per se, for example by mixing the active compounds with at least one customary extender, solvent or diluent, emulsifier, dispersant, and/or binder or fixative, wetting agent, water repellent, if appropriate desiccants and UV stabilizers and, if appropriate, dyes and pigments, defoamers, preservatives, secondary thickeners, adhesives, gibberellins and also further processing auxiliaries.

The compositions according to the invention include not only formulations which are already ready for use and can be applied with a suitable apparatus to the plant or the seed, but also commercial concentrates which have to be diluted with water prior to use. The formulations are prepared either in suitable plants or else before or during application.

The active compounds according to the invention can be present as such or in their (commercial) formulations and in the use forms prepared from these formulations as a mixture with other (known) active compounds, such as insecticides, attractants, sterilants, bactericides, acaricides, nematicides, fungicides, growth regulators, herbicides, fertilizers, safeners and/or semiochemicals.

The treatment according to the invention of the plants and plant parts with the active compounds or compositions is carried out directly or by action on their surroundings, habitat or storage space using customary treatment methods, for example by dipping, spraying, atomizing, irrigating, evaporating, dusting, fogging, broadcasting, foaming, painting, spreading-on, watering (drenching), drip irrigating and, in the case of propagation material, in particular in the case of seeds, furthermore as a powder for dry seed treatment, a solution for wet seed treatment, a water-soluble powder for slurry treatment, by incrusting, by coating with one or more coats, etc. It is furthermore possible to apply the active compounds by the ultra-low volume method or to inject the active compound preparation or the active compound itself into the soil.

The invention furthermore includes a method for treating seed.

The invention furthermore relates to seed which has been treated in accordance with one of the methods described in the previous paragraph. The seeds according to the invention are used in methods for the protection of seed from undesirable fungi. In these methods, seed treated with at least one active compound according to the invention is employed.

The active compounds or compositions according to the invention are also suitable for treating seed. A large part of the damage to crop plants caused by harmful organisms is triggered by the infection of the seed during storage or after sowing both during and after germination of the plant. This phase is particularly critical since the roots and shoots of the growing plant are particularly sensitive, and even small damage may result in the death of the plant. Accordingly, there is great interest in protecting the seed and the germinating plant by using appropriate compositions.

The control of phytopathogenic fungi by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with, or at least reduce considerably, the additional application of crop protection agents after sowing or after emergence of the plants. It is furthermore desirable to optimize the amount of active compound employed in such a way as to provide optimum protection for the seed and the germinating plant from attack by phytopathogenic fungi, but without damaging the plant itself by the active compound employed. In particular, methods for the treatment of seed should also take into consideration the intrinsic fungicidal properties of transgenic plants in order to achieve optimum protection of the seed and the germinating plant with a minimum of crop protection agents being employed.

The present invention therefore also relates to a method for the protection of seed and germinating plants, from attack by phytopathogenic fungi, by treating the seed with a composition according to the invention. The invention also relates to the use of the compositions according to the invention for treating seed for protecting the seed and the germinating plant against phytopathogenic fungi. Furthermore, the invention relates to seed treated with a composition according to the invention for protection against phytopathogenic fungi.

The control of phytopathogenic fungi which damage plants post-emergence is carried out primarily by treating the soil and the above-ground parts of plants with crop protection agents. Owing to the concerns regarding a possible impact of the crop protection agents on the environment and the health of humans and animals, there are efforts to reduce the amount of active compounds applied.

One of the advantages of the present invention is that the particular systemic properties of the active compounds and compositions according to the invention mean that treatment of the seed with these active compounds and compositions not only protects the seed itself, but also the resulting plants after emergence, from phytopathogenic fungi. In this manner, the immediate treatment of the crop at the time of sowing or shortly thereafter can be dispensed with.

It is also considered to be advantageous that the active compounds or compositions according to the invention can be used in particular also for transgenic seed where the plant growing from this seed is capable of expressing a protein which acts against pests. By treating such seed with the active compounds or compositions according to the invention, even by the expression of the, for example, insecticidal protein, certain pests may be controlled. Surprisingly, a further synergistic effect may be observed here, which additionally increases the effectiveness of the protection against attack by pests.

The compositions according to the invention are suitable for protecting seed of any plant variety which is employed in agriculture, in the greenhouse, in forests or in horticulture and viticulture. In particular, this takes the form of seed of cereals (such as wheat, barley, rye, triticale, sorghum/millet and oats), maize, cotton, soya beans, rice, potatoes, sunflower, bean, coffee, beet (for example sugar beet and fodder beet), peanut, oilseed rape, poppy, olive, coconut, cacao, sugar cane, tobacco, vegetables (such as tomato, cucumbers, onions and lettuce), turf and ornamentals (see also hereinbelow). The treatment of the seed of cereals (such as wheat, barley, rye, triticale and oats), maize and rice is of particular importance.

As also described further below, the treatment of transgenic seed with the active compounds or compositions according to the invention is of particular importance. This refers to the seed of plants containing at least one heterologous gene which allows the expression of a polypeptide or protein having insecticidal properties. The heterologous gene in transgenic seed can originate, for example, from microorganisms of the species Bacillus, Rhizobium, Pseudomonas, Serratia, Trichoderma, Clavibacter, Glomus or Gliocladium. Preferably, this heterologous gene is from Bacillus sp., the gene product having activity against the European corn borer and/or the Western corn rootworm. Particularly preferably, the heterologous gene originates from Bacillus thuringiensis.

Within the context of the present invention, the composition according to the invention is applied to the seed either alone or in a suitable formulation. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits. Thus, it is possible to use, for example, seed which has been harvested, cleaned and dried to a moisture content of less than 15% by weight. Alternatively, it is also possible to use seed which, after drying, has been treated, for example, with water and then dried again.

When treating the seed, care must generally be taken that the amount of the composition according to the invention applied to the seed and/or the amount of further additives is chosen in such a way that the germination of the seed is not adversely affected, or that the resulting plant is not damaged. This must be borne in mind in particular in the case of active compounds which can have phytotoxic effects at certain application rates.

The compositions according to the invention can be applied directly, i.e. without containing any other components and undiluted. In general, it is preferred to apply the compositions to the seed in the form of a suitable formulation. Suitable formulations and methods for treating seed are known to the person skilled in the art and are described, for example, in the following documents: U.S. Pat. No. 4,272,417 A, U.S. Pat. No. 4,245,432, U.S. Pat. No. 4,808,430, U.S. Pat. No. 5,876,739, US 2003/0176428 μl, WO 2002/080675, WO 2002/028186.

The active compounds which can be used in accordance with the invention can be converted into the customary seed-dressing formulations, such as solutions, emulsions, suspensions, powders, foams, slurries or other coating compositions for seed, and also ULV formulations.

These formulations are prepared in a known manner, by mixing the active compounds with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. In this context, not only pigments, which are sparingly soluble in water, but also dyes, which are soluble in water, may be used. Examples include the dyes known by the names Rhodamine B, C.I. Pigment Red 112 and C.I. Solvent Red 1.

Suitable wetting agents which may be present in the seed-dressing formulations which can be used in accordance with the invention are all substances which promote wetting and which are conventionally used for the formulation of agrochemical active compounds. Preference is given to using alkylnaphthalenesulphonates, such as diisopropyl or diisobutylnaphthalenesulphonates.

Suitable dispersants and/or emulsifiers which may be present in the seed-dressing formulations which can be used in accordance with the invention are all nonionic, anionic and cationic dispersants conventionally used for the formulation of agrochemical active compounds. Preference is given to using nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristryrylphenol polyglycol ether, and their phosphated or sulphated derivatives. Suitable anionic dispersants are, in particular, lignosulphonates, polyacrylic acid salts and arylsulphonate/formaldehyde condensates.

Antifoams which may be present in the seed-dressing formulations which can be used in accordance with the invention are all foam-inhibiting substances conventionally used for the formulation of agrochemical active compounds. Silicone antifoams and magnesium stearate can preferably be used.

Preservatives which may be present in the seed-dressing formulations which can be used in accordance with the invention are all substances which can be employed for such purposes in agrochemical compositions. Dichlorophene and benzyl alcohol hemiformal may be mentioned by way of example.

Secondary thickeners which may be present in the seed-dressing formulations which can be used in accordance with the invention are all substances which can be employed for such purposes in agrochemical compositions. Cellulose derivatives, acrylic acid derivatives, xanthan, modified clays and finely divided silica are preferred.

Adhesives which may be present in the seed-dressing formulations which can be used in accordance with the invention are all customary binders which can be employed in seed-dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schädlings-bekämpfungsmittel” [Chemistry of the Crop Protection Compositions and Pesticides], vol. 2, Springer Verlag, 1970, p. 401-412).

The seed-dressing formulations which can be used in accordance with the invention can be employed for the treatment of a wide range of seed, including the seed of transgenic plants, either directly or after previously having been diluted with water. In this context, additional synergistic effects may also occur in cooperation with the substances formed by expression.

All mixers which can conventionally be employed for the seed-dressing operation are suitable for treating seed with the seed-dressing formulations which can be used in accordance with the invention or with the preparations prepared therefrom by addition of water. Specifically, a procedure is followed during the seed-dressing operation in which the seed is placed into a mixer, the specific desired amount of seed-dressing formulations, either as such or after previously having been diluted with water, is added, and everything is mixed until the formulation is distributed uniformly on the seed. If appropriate, this is followed by a drying process.

The active compounds or compositions according to the invention have a potent fungicidal activity and can be employed for controlling undesirable fungi in crop protection and in the protection of materials.

The compounds according to the invention can be employed in crop protection for controlling Plasmodiophoromycetes, Oomycetes, Chytridiomycetes, Zygomycetes, Ascomycetes, Basidiomycetes and Deuteromycetes.

The fungicidal compositions according to the invention can be used for the curative or protective control of phytopathogenic fungi. Accordingly, the invention also relates to curative and protective methods for controlling phytopathogenic fungi using the active compounds or compositions according to the invention, which are applied to the seed, the plant or plant parts, the fruit or the soil in which the plants grow.

The compositions according to the invention for controlling phytopathogenic fungi in crop protection comprise an effective, but non-phytotoxic amount of the active compounds according to the invention. “Effective, but non-phytotoxic amount” means an amount of the composition according to the invention which is sufficient to control the fungal disease of the plant in a satisfactory manner or to eradicate the fungal disease completely, and which, at the same time, does not cause any significant symptoms of phytotoxicity. In general, this application rate may vary within a relatively wide range. It depends on a plurality of factors, for example on the fungus to be controlled, the plant, the climatic conditions and the ingredients of the compositions according to the invention.

The fact that the active compounds are well tolerated by plants at the concentrations required for controlling plant diseases allows the treatment of above-ground parts of plants, of propagation stock and seeds, and of the soil.

All plants and plant parts can be treated in accordance with the invention. Plants are understood here to mean all plants and plant populations, such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. Parts of plants also include harvested plants and vegetative and generative propagation material, for example seedlings, tubers, rhizomes, cuttings and seeds.

The active compounds according to the invention are suitable for the protection of plants and plant organs, for increasing the harvest yields, for improving the quality of the harvested crop, while being well tolerated by plants, having favourable toxicity to warm-blooded species and being environmentally friendly. They may be preferably employed as crop protection agents. They are active against normally sensitive and resistant species and against all or some stages of development.

The following plants may be mentioned as plants which can be treated according to the invention: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actinidaceae sp., Lauraceae sp., Musaceae sp. (for example banana plants and banana plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leeks, onions), Papilionaceae sp. (for example peas); major crop plants such as Gramineae sp. (for example maize, turf, cereals such as wheat, rye, rice, barley, oats, millet and triticale), Poaceae sp. (for example sugar cane), Asteraceae sp. (for example sunflower), Brassicaceae sp. (for example white cabbage, red cabbage, broccoli, cauliflower, Brussels sprouts, pak choi, kohlrabi, small radishes, and also oilseed rape, mustard, horseradish and cress), Fabacae sp. (for example beans, peanuts), Papilionaceae sp. (for example soya bean), Solanaceae sp. (for example potatoes), Chenopodiaceae sp. (for example sugar beet, fodder beet, Swiss chard, beetroot); useful plants and ornamental plants in gardens and forests; and in each case genetically modified types of these plants.

As already mentioned above, it is possible to treat all plants and their parts according to the invention. In a preferred embodiment, wild plant species and plant cultivars, or those obtained by conventional biological breeding, such as crossing or protoplast fusion, and parts thereof, are treated. In a further preferred embodiment, transgenic plants and plant cultivars obtained by genetic engineering, if appropriate in combination with conventional methods (Genetically Modified Organisms), and parts thereof are treated. The term “parts” or “parts of plants” or “plant parts” has been explained above. Particularly preferably, plants of the plant cultivars which are in each case commercially available or in use are treated according to the invention. Plant cultivars are to be understood as meaning plants having new properties (“traits”) and which have been obtained by conventional breeding, by mutagenesis or by recombinant DNA techniques. They can be cultivars, varieties, bio- or genotypes.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNAi technology [RNA interference]). A heterologous gene that is located in the genome is also called a transgene. A transgene that is defined by its particular location in the plant genome is called a transformation or transgenic event.

Depending on the plant species or plant varieties, their location and growth conditions (soils, climate, vegetation period, diet), the treatment according to the invention may also result in superadditive (“synergistic”) effects. For example, the following effects exceeding the effects actually to be expected are possible: reduced application rates and/or a widening of the activity spectrum and/or an increase in the activity of the active compounds and compositions which can be used according to the invention, better plant growth, increased tolerance to high or low temperatures, increased tolerance to drought or to water or soil salt content, increased flowering performance, easier harvesting, accelerated maturation, higher harvest yields, bigger fruits, larger plant height, greener leaf colour, earlier flowering, higher quality and/or a higher nutritional value of the harvested products, higher sugar concentration within the fruits, better storage stability and/or processability of the harvested products.

At certain application rates, the active compound combinations according to the invention may also have a strengthening effect in plants. Accordingly, they are suitable for mobilizing the defence system of the plant against attack by unwanted phytopathogenic fungi and/or microorganisms and/or viruses. This may, if appropriate, be one of the reasons for the enhanced activity of the combinations according to the invention, for example against fungi. Plant-strengthening (resistance-inducing) substances are to be understood as meaning, in the present context, also those substances or combinations of substances which are capable of stimulating the defence system of plants in such a way that, when subsequently inoculated with unwanted phytopathogenic fungi, the treated plants display a substantial degree of resistance to these unwanted phytopathogenic fungi. Thus, the substances according to the invention can be employed for protecting plants against attack by the above-mentioned pathogens within a certain period of time after the treatment. The period within which protection is brought about generally extends from 1 to 10 days, preferably 1 to 7 days, after the treatment of the plants with the active compounds.

Plants and plant varieties which are preferably treated according to the invention include all plants which have genetic material which imparts particularly advantageous, useful traits to these plants (whether obtained by breeding and/or biotechnological means).

Plants and plant varieties which are also preferably treated according to the invention are resistant against one or more biotic stress factors, i.e. said plants have a better defence against animal and microbial pests, such as against nematodes, insects, mites, phytopathogenic fungi, bacteria, viruses and/or viroids.

Plants and plant varieties which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or hybrid effect, which results in generally higher yield, vigour, health and resistance towards biotic to and abiotic stress factors. Such plants are typically made by crossing an inbred male-sterile parent line (the female parent) with another inbred male-fertile parent line (the male parent). Hybrid seed is typically harvested from the male-sterile plants and sold to growers. Male-sterile plants can sometimes (e.g. in maize) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described for Brassica species. However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar.

Plants or plant varieties (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium, the CP4 gene of the bacterium Agrobacterium sp., the genes encoding a petunia EPSPS, a tomato EPSPS, or an Eleusine EPSPS. It can also be a mutated EPSPS. Glyphosate-tolerant plants can also be obtained by expressing a gene that encodes a glyphosate oxidoreductase enzyme. Glyphosate-tolerant plants can also be obtained by expressing a gene that encodes a glyphosate acetyltransferase enzyme. Glyphosate-tolerant plants can also be obtained by selecting plants containing naturally occurring mutations of the abovementioned genes.

Other herbicide-resistant plants are for example plants which have been made tolerant to herbicides inhibiting the enzyme glutamine synthase, such as bialaphos, phosphinothricin or glufosinate. Such plants can be obtained by expressing an enzyme detoXifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species for example). Plants expressing an exogenous phosphinothricin acetyltransferase have been described.

Further herbicide-tolerant plants are also plants that have been made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD inhibitors can be transformed with a gene encoding a naturally occurring resistant HPPD enzyme, or a gene encoding a mutated HPPD enzyme. Tolerance to HPPD inhibitors can also be obtained by transforming plants with genes encoding certain enzymes enabling the formation of homogentisate despite the inhibition of the native HPPD enzyme by the HPPD inhibitor. Tolerance of plants to HPPD inhibitors can also be improved by transforming plants with a gene encoding an enzyme prephenate dehydrogenase in addition to a gene encoding an HPPD-tolerant enzyme.

Further herbicide-resistant plants are plants that have been made tolerant to acetolactate synthase (ALS) inhibitors. Known ALS inhibitors include, for example, sulphonylurea, imidazolinone, triazolopyrimidines, pyrimidinyl oxy(thio)benzoates, and/or sulphonylaminocarbonyltriazolinone herbicides. Different mutations in the ALS enzyme (also known as acetohydroxy acid synthase, AHAS) are known to confer tolerance to different herbicides and groups of herbicides. The production of sulphonylurea-tolerant plants and imidazolinone-tolerant plants has been described in the international publication WO 1996/033270. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:

1) an insecticidal crystal protein from Bacillus thuringiensis or an insecticidal portion thereof, such as the insecticidal crystal proteins listed online at: http://www.lifesci.sussex.ac.uk/Home/Neil_Crickmore/Bt/, or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein as Bacillus thuringiensis or a portion thereof, such as the binary toxin made up of the Cy34 and Cy35 crystal proteins; or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bbl protein in maize events MON863 or MON88017, or the Cry3A protein in maize event MIR604; or
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and V1P2A proteins;
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:

a. plants which contain a transgene capable of reducing the expression and/or the activity of the poly(ADP-ribose)polymerase (PARP) gene in the plant cells or plants;
b. plants which contain a stress tolerance-enhancing transgene capable of reducing the expression and/or the activity of the PARG-encoding genes of the plants or plant cells;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyltransferase, nicotinamide adenine dinucleotide synthetase or nicotinamide phosphoribosyltransferase.

Plants or plant varieties (obtained by plant biotechnology methods such as genetic engineering) which may also be treated according to the invention show altered quantity, quality and/or storage stability of the harvested product and/or altered properties of specific ingredients of the harvested product such as, for example:

1) Transgenic plants which synthesize a modified starch which is altered with respect to its chemophysical traits, in particular the amylose content or the amylose/amylopectin ratio, the degree of branching, the average chain length, the distribution of the side chains, the viscosity behaviour, the gel resistance, the grain size and/or grain morphology of the starch in comparison to the synthesized starch in wild-type plant cells or plants, such that this modified starch is better suited for certain applications.
2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, plants which produce alpha-1,4-glucans, plants which produce alpha-1,6-branched alpha-1,4-glucans, and plants producing alternan.
3) Transgenic plants which produce hyaluronan.

Plants or plant varieties (obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes;
b) plants, such as cotton plants, which contain an altered form of rsw2 or rsw3 homologous nucleic acids;
c) plants, such as cotton plants, with an increased expression of sucrose phosphate synthase;
d) plants, such as cotton plants, with an increased expression of sucrose synthase;
e) plants, such as cotton plants, wherein the timing of the plasmodesmatal gating at the basis of the fibre cell is altered, for example through downregulation of fibre-selective β-1,3-glucanase;
f) plants, such as cotton plants, which have fibres with altered reactivity, for example through the expression of the N-acetylglucosaminetransferase gene including nodC and chitin synthase genes.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:

a) plants, such as oilseed rape plants, which produce oil having a high oleic acid content;
b) plants, such as oilseed rape plants, which produce oil having a low linolenic acid content;
c) plants, such as oilseed rape plants, which produce oil having a low level of saturated fatty acids.

Particularly useful transgenic plants which may be treated according to the invention are plants which comprise one or more genes which encode one or more toxins and are the transgenic plants available under the following trade names: YIELD GARD® (for example maize, cotton, soybeans), KnockOut® (for example maize), BiteGard® (for example maize), BT-Xtra® (for example maize), StarLink® (for example maize), Bollgard@ (cotton), Nucotn® (cotton), Nucotn 33B® (cotton), NatureGard® (for example maize), Protecta® and New-Leaf® (potato). Examples of herbicide-tolerant plants which may be mentioned are maize varieties, cotton varieties and soya bean varieties which are available under the following trade names: Roundup Ready® (tolerance to glyphosate, for example maize, cotton, soya beans), Liberty Link® (tolerance to phosphinothricin, for example oilseed rape), IMOD (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, and that are listed for example in the databases for various national or regional regulatory agencies (see for example http://gmoinfo.jrc.it/gmp_browse.aspx and http://www.agbios.com/dbase.php).

The active compounds according to the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as crop protection agents. They are active against normally sensitive and resistant species and against all or some stages of development. The abovementioned pests include:

From the phylum Mollusca, for example from the class of the Lamellibranchiata, for example Dreissena spp.
From the class of the Gastropoda, for example, Arlon spp., Biomphalaria spp., Bulinus spp., Deroceras spp., Galba spp., Lymnaea spp., Oncomelania spp., Pomacea spp., Succinea spp.
From the phylum Arthropoda, for example from the order of the Isopoda, for example Armadillidium vulgare, Oniscus asellus, Porcellio scaber.
From the class of the Arachnida, for example, Acarus spp., Aceria sheldoni, Aculops spp., Aculus spp., Amblyomma spp., Amphitetranychus viennensis, Argas spp., Boophilus spp., Brevipalpus spp., Bryobia praetiosa, Centruroides spp., Chorioptes spp., Dermanyssus gallinae, Dermatophagoides pteronyssius, Dermatophagoides farinae, Dermacentor spp., Eotetranychus spp., Epitrimerus pyri, Eutetranychus spp., Eriophyes spp., Halotydeus destructor, Hemitarsonemus spp., Hyalomma spp., Ixodes spp., Latrodectus spp., Loxosceles spp., Metatetranychus spp., Nuphersa spp., Oligonychus spp., Ornithodorus spp., Ornithonyssus spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vaejovis spp., Vasates lycopersici.
From the order of the Symphyla, for example, Scutigerella spp.
From the order of the Chilopoda, for example, Geophilus spp., Scutigera spp.
From the order of the Collembola, for example, Onychiurus armatus.
From the order of the Diplopoda, for example, Blaniulus guttulatus.
From the order of the Zygentoma, for example, Lepisma saccharin, Thermobia domestica.
From the order of the Orthoptera, for example, Acheta domesticus, Blatta orientalis, Blattella germanica, Dichroplus spp., Gryllotalpa spp., Leucophaea maderae, Locusta spp., Melanoplus spp., Periplaneta spp., Pulex irritans, Schistocerca gregaria, Supella longipalpa.
From the order of the Isoptera, for example, Coptotermes spp., Comitermes cumulans, Cryptotermes spp., Incisitermes spp., Microtermes obesi, Odontotermes spp., Reticulitermes spp.
From the order of the Heteroptera, for example, Anasa tristis, Antestiopsis spp., Boisea spp., Blissus spp., Calocoris spp., Campylomma livida, Cavelerius spp., Cimex lectularius, Collaria spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Monalonion atratum, Nezara spp., Oebalus spp., Pentomidae, Piesma quadrata, Piezodorus spp., Psallus spp., Pseudacysta persea, Rhodnius spp., Sahlbergella singularis, Scaptocoris castanea, Scotinophora spp., Stephanitis nashi, Tibraca spp., Triatoma spp.
From the order of the Anoplura (Phthiraptera), for example, Damalinia spp., Haematopinus spp., Linognathus spp., Pediculus spp., Ptirus pubis, Trichodectes spp.
From the order of the Homoptera, for example, Acyrthosipon spp., Acrogonia spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Cameocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Drosicha spp., Dysaphis spp., Dysmicoccus spp., Empoasca spp., Eriosoma spp., Erythroneura spp., Euscelis bilobatus, Ferrisia spp., Geococcus coffeae, Hieroglyphus spp., Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva spp., Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes spp., Trioza spp., Typhlocyba spp., Unaspis spp., Viteus vitifolii, Zygina spp.
From the order of the Coleoptera, for example, Acalymma vittatum, Acanthoscelides obtectus, Adoretus spp., Agelastica alni, Agriotes spp., Alphitobius diaperinus, Amphimallon solstitialis, Anobium punctatum, Anoplophora spp., Anthonomus spp., Anthrenus spp., Apion spp., Apogonia spp., Atomaria spp., Attagenus spp., Bruchidius obtectus, Bruchus spp., Cassida spp., Cerotoma trifurcata, Ceutorrhynchus spp., Chaetocnema spp., Cleonus mendicus, Conoderus spp., Cosmopolites spp., Costelytra zealandica, Ctenicera spp., Curculio spp., Cryptorhynchus lapathi, Cylindrocopturus spp., Dermestes spp., Diabrotica spp., Dichocrocis spp., Diloboderus spp., Epilachna spp., Epitrix spp., Faustinus spp., Gibbium psylloides, Hellula undalis, Heteronychus arator, Heteronyx spp., Hylamorpha elegans, Hylotrupes bajulus, Hypera postica, Hypothenemus spp., Lachnostema consanguinea, Lema spp., Leptinotarsa decemlineata, Leucoptera spp., Lissorhoptrus oryzophilus, Lixus spp., Luperodes spp., Lyctus spp., Megascelis spp., Melanotus spp., Meligethes aeneus, Melolontha spp., Migdolus spp., Monochamus spp., Naupactus xanthographus, Niptus hololeucus, Oryctes rhinoceros, Oryzaephilus surinamensis, Oryzaphagus oryzae, Otiorrhynchus spp., Oxycetonia jucunda, Phaedon cochleariae, Phyllophaga spp., Phyllotreta spp., Popillia japonica, Premnotrypes spp., Prostephanus truncatus, Psylliodes spp., Ptinus spp., Rhizobius ventralis, Rhizopertha dominica, Sitophilus spp., Sphenophorus spp., Stegobium paniceum, Sternechus spp., Symphyletes spp., Tanymecus spp., Tenebrio molitor, Tribolium spp., Trogoderma spp., Tychius spp., Xylotrechus spp., Zabrus spp.
From the order of the Hymenoptera, for example, Acromyrmex spp., Athalia spp., Atta spp., Diprion spp., Hoplocampa spp., Lasius spp., Monomorium pharaonis, Solenopsis invicta, Tapinoma spp., Vespa spp.
From the order of the Lepidoptera, for example, Acronicta major, Adoxophyes spp., Aedia leucomelas, Agrotis spp., Alabama spp., Amyelois transitella, Anarsia spp., Anticarsia spp., Argyroploce spp., Barathra brassicae, Borbo cinnara, Bucculatrix thurberiella, Bupalus piniarius, Busseola spp., Cacoecia spp., Caloptilia theivora, Capua reticulana, Carpocapsa pomonella, Carposina niponensis, Chematobia brumata, Chilo spp., Choristoneura spp., Clysia ambiguella, Cnaphalocerus spp., Cnephasia spp., Conopomorpha spp., Conotrachelus spp., Copitarsia spp., Cydia spp., Dalaca noctuides, Diaphania spp., Diatraea saccharalis, Earias spp., Ecdytolopha aurantium, Elasmopalpus lignosellus, Eldana saccharin, Ephestia spp., Epinotia spp., Epiphyas postvittana, Etiella spp., Eulia spp., Eupoecilia ambiguella, Euproctis spp., Euxoa spp., Feltia spp., Galleria mellonella, Gracillaria spp., Grapholitha spp., Hedylepta spp., Helicoverpa spp., Heliothis spp., Hofmannophila pseudospretella, Homoeosoma spp., Homona spp., Hyponomeuta padella, Kakivoria flavofasciata, Laphygma spp., Laspeyresia molesta, Leucinodes orbonalis, Leucoptera spp., Lithocolletis spp., Lithophane antennata, Lobesia spp., Loxagrotis albicosta, Lymantria spp., Lyonetia spp., Malacosoma neustria, Maruca testulalis, Mamestra brassicae, Mocis spp., Mythimna separata, Nymphula spp., Oiketicus spp., Oria spp., Orthaga spp., Ostrinia spp., Oulema oryzae, Panolis flammea, Pamara spp., Pectinophora spp., Perileucoptera spp., Phthorimaea spp., Phyllocnistis citrella, Phyllonorycter spp., Pieris spp., Platynota stultana, Plodia interpunctella, Plusia spp., Plutella xylostella, Prays spp., Prodenia spp., Protoparce spp., Pseudaletia spp., Pseudoplusia includens, Pyrausta nubilalis, Rachiplusia nu, Schoenobius spp., Scirpophaga spp., Scotia segetum, Sesamia spp., Sparganothis spp.; Spodoptera spp., Stathntopoda spp., Stomopteryx subsecivella, Synanthedon spp., Tecia solanivora, Thermesia gemmatalis, Tinea pellionella, Tineola bisselliella, Tortrix spp., Trichophaga tapetzella, Trichoplusia spp., Tuta absoluta, Virachola spp.
From the order of the Diptera, for example, Aedes spp., Agromyza spp., Anastrepha spp., Anopheles spp., Asphondylia spp., Bactrocera spp., Bibio hortulanus, Calliphora erythrocephala, Ceratitis capitata, Chironomus spp., Chrysomyia spp., Chrysops spp., Cochliomyia spp., Contarinia spp., Cordylobia anthropophaga, Culex spp., Culicoides spp., Culiseta spp., Cuterebra spp., Dacus oleae, Dasyneura spp., Delia spp., Dermatobia hominis, Drosophila spp., Echinocnemus spp., Fannia spp., Gasterophilus spp., Glossina spp., Haematopta spp., Hydrellia spp., Hylemyia spp., Hyppobosca spp., Hypoderma spp., Liriomyza spp., Lucilia spp., Lutzomia spp., Mansonia spp., Musca spp., Nezara spp., Oestrus spp., Oscinella frit, Pegomyia spp., Phlebotomus spp., Phorbia spp., Phormia spp., Prodiplosis spp., Psila rosae, Rhagoletis spp., Sarcophaga spp., Simulium spp, Stomoxys spp., Tabanus spp., Tannia spp., Tetanops spp., Tipula spp.
From the order of the Thysanoptera, for example, Anaphothrips obscurus, Baliothrips biformis, Drepanothris reuteri, Enneothrips flavens, Frankliniella spp., Heliothrips spp., Hercinothrips femoralis, Rhipiphorothrips cruentatus, Scirtothrips spp., Taeniothrips cardamoni, Thrips spp.
From the order of the Siphonaptera, for example, Ceratophyllus spp., Ctenocephalides spp., Tunga penetrans, Xenopsylla cheopis.
From the phyla of the Plathelminths and Nematodes as animal parasites, for example from the class of the Helminths, for example Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp., Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Strongyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.
From the phylum of the Nematodes as plant pests, for example, Aphelenchoides spp., Bursaphelenchus spp., Ditylenchus spp., Globodera spp., Heterodera spp., Longidorus spp., Meloidogyne spp., Pratylenchus spp., Radopholus similis, Trichodorus spp., Tylenchulus semipenetrans, Xiphinema spp.
From the subphylum of the Protozoa, for example Eimeria.

When used as insecticides, the active compounds according to the invention can furthermore be present in their commercially available formulations and in the use forms, prepared from these formulations, as a mixture with synergistic agents. Synergists are compounds which increase the action of the active compounds, without it being necessary for the synergist added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.
From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.
From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp. and Melophagus spp.
From the order of the Siphonapterida, for example Pulex spp., Ctenocephalides spp., Xenopsylla spp. and Ceratophyllus spp.
From the order of the Heteropterida, for example, Cimex spp., Triatoma spp., Rhodnius spp. and Panstrongylus spp.
From the order of the Blattarida, for example Blatta orientalis, Periplaneta americana, Blattela germanica and Supella spp.
From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Steprnostoma spp. and Varroa spp.
From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for livestock, poultry, domestic animals and the like, the active compounds of the formula (I) can be used as formulations (for example powders, emulsions, flowables) comprising the active compounds in an amount of 1 to 80% by weight, either directly or after 100 to 10 000 fold dilution, or they may be used as a chemical bath.

Moreover, in the protection of materials, the active compounds or compositions according to the invention can be employed for protecting industrial materials against attack and destruction by unwanted microorganisms, such as, for example, fungi.

Industrial materials in the present context are understood to mean inanimate materials which have been prepared for use in industry. For example, industrial materials which are intended to be protected by active compounds according to the invention from fungal change or destruction can be adhesives, sizes, paper, wallpaper, and board, textiles, carpets, leather, wood, paints and plastic articles, cooling lubricants and other materials which can be infected with, or destroyed by, microorganisms. Parts of production plants and buildings, for example cooling-water circuits, cooling and heating systems and ventilation and air-conditioning units, which may be impaired by the proliferation of microorganisms may also be mentioned within the scope of the materials to be protected. Industrial materials which may be mentioned within the scope of the present invention are preferably adhesives, sizes, paper and board, leather, wood, paints, cooling lubricants and heat-transfer liquids, particularly preferably wood. The active compounds or compositions according to the invention may prevent disadvantageous effects, such as rotting, decay, discoloration, decoloration or formation of mould. Moreover, the compounds according to the invention can be employed for protecting objects which come into contact with saltwater or brackish water, in particular hulls, screens, nets, buildings, moorings and signalling systems, against fouling.

It has furthermore been found that the compounds according to the invention have a strong insecticidal action against insects which destroy industrial materials.

The following insects may be mentioned as examples and as preferred—but without limitation: beetles, such as Hylotrupes bajulus, Chlorophorus pilosis, Anobium punctatum, Xestobium rufovillosum, Ptilinus pecticornis, Dendrobium pertinex, Emobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus; Hymenoptera, such as Sirex juvencus, Urocerus gigas, Urocerus gigas taignus, Urocerus augur, termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus; bristletails, such as Lepisma saccharina.

Industrial materials in the present connection are to be understood as meaning non-living materials, such as, preferably, plastics, adhesives, sizes, papers and cards, leather, wood, processed wood products and coating compositions.

The ready-to-use compositions may optionally also comprise other insecticides, and optionally one or more fungicides.

With respect to possible additional partners for mixing, reference is made to insecticides and fungicides. Moreover, the compounds according to the invention can be employed for protecting objects which come into contact with saltwater or brackish water, in particular hulls, screens, nets, buildings, moorings and signalling systems, against fouling.

Furthermore, the compounds according to the invention can be used alone or in combinations with other active compounds as antifouling compositions.

The active compounds are also suitable for controlling animal pests in the domestic sector, in the hygiene sector and in the protection of stored products, especially insects, arachnids and mites, which are found in enclosed spaces, for example homes, factory halls, offices, vehicle cabins and the like. They can be used to control these pests alone or in combination with other active compounds and auxiliaries in domestic insecticide products. They are effective against sensitive and resistant species, and against all developmental stages. These pests include:

From the order of the Scorpionidea, for example, Buthus occitanus.
From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.
From the order of the Araneae, for example, Aviculariidae, Araneidae.
From the order of the Opiliones, for example, Pseudoscorpiones chelifer, Pseudoscorpiones cheiridium, Opiliones phalangium.
From the order of the Isopoda, for example, Oniscus asellus, Porcellio scaber.
From the order of the Diplopoda, for example, Blaniulus guttulatus, Polydesmus spp.
From the order of the Chilopoda, for example, Geophilus spp.
From the order of the Zygentoma, for example, Ctenolepisma spp., Lepisma saccharina, Lepismodes inquilinus.
From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.
From the order of the Saltatoria, for example, Acheta domesticus.
From the order of the Dermaptera, for example, Forficula auricularia.
From the order of the Isopters, for example, Kalotermes spp., Reticulitermes spp.
From the order of the Psocoptera, for example, Lepinatus spp.; Liposcelis spp.
From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.
From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga camaria, Simulium spp., Stomoxys calcitrans, Tipula paludosa.
From the order of the Lepidoptera, for example, Achroia grisella, Galleria mellonella, Plodia interpunctella, Tinea cloacella, Tinea pellionella, Tineola bisselliella.
From the order of the Siphonaptera, for example, Ctenocephalides canis, Ctenocephalides felis, Pulex irritans, Tunga penetrans, Xenopsylla cheopis.
From the order of the Hymenoptera, for example, Camponotus herculeanus, Lasius fuliginosus, Lasius niger, Lasius umbratus, Monomorium pharaonis, Paravespula spp., Tetramorium caespitum.
From the order of the Anoplura, for example, Pediculus humanus capitis, Pediculus humanus corporis, Pemphigus spp., Phylloera vastatrix, Phthirus pubis.
From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

The method according to the invention for controlling unwanted fungi can also be employed for protecting storage goods. Here, storage goods are to be understood as meaning natural substances of vegetable or animal origin or processed products thereof of natural origin, for which long-term protection is desired. Storage goods of vegetable origin, such as, for example, plants or plant parts, such as stems, leaves, tubers, seeds, fruits, grains, can be protected freshly harvested or after processing by (pre)drying, moistening, comminuting, grinding, pressing or roasting. Storage goods also include timber, both unprocessed, such as construction timber, electricity poles and barriers, or in the form of finished products, such as furniture. Storage goods of animal origin are, for example, hides, leather, furs and hairs. The active compounds according to the invention may prevent disadvantageous effects, such as rotting, decay, discoloration, decoloration or formation of mould.

Some pathogens of fungal diseases which can be treated according to the invention may be mentioned by way of example, but not by way of limitation:

caused by powdery mildew pathogens, such as, for example, Blumeria species, such as, for example, Blumeria graminis; Podosphaera species, such as, for example, Podosphaera leucotricha; Sphaerotheca species, such as, for example, Sphaerotheca fuliginea; Uncinula species, such as, for example, Uncinula necator;
diseases caused by rust disease pathogens, such as, for example, Gymnosporangium species, such as, for example, Gymnosporangium sabinae; Hemileia species, such as, for example, Hemileia vastatrix; Phakopsora species, such as, for example, Phakopsora pachyrhizi and Phakopsora meibomiae; Puccinia species, such as, for example, Puccinia recondita or Puccinia triticina; Uromyces species, such as, for example, Uromyces appendiculatus;
diseases caused by pathogens from the group of the Oomycetes, such as, for example, Bremia species, such as, for example, Bremia lactucae; Peronospora species, such as, for example, Peronospora pisi or P. brassicae; Phytophthora species, such as, for example, Phytophthora infestans; Plasmopara species, such as, for example, Plasmopara viticola; Pseudoperonospora species, such as, for example, Pseudoperonospora humuli or Pseudoperonospora cubensis; Pythium species, such as, for example, Pythium ultimum;
leaf blotch diseases and leaf wilt diseases caused, for example, by Alternaria species, such as, for example, Altemaria solani; Cercospora species, such as, for example, Cercospora beticola; Cladiosporium species, such as, for example, Cladiosporium cucumerinum; Cochliobolus species, such as, for example, Cochliobolus sativus (conidia form: Drechslera, syn: Helminthosporium); Colletotrichum species, such as, for example, Colletotrichum lindemuthanium; Cycloconium species, such as, for example, Cycloconium oleaginum; Diaporthe species, such as, for example, Diaporthe citri; Elsinoe species, such as, for example, Elsinoe fawcettii; Gloeosporium species, such as, for example, Gloeosporium laeticolor; Glomerella species, such as, for example, Glomerella cingulate; Guignardia species, such as, for example, Guignardia bidwelli; Leptosphaeria species, such as, for example, Leptosphaeria maculans; Magnaporthe species, such as, for example, Magnaporthe grisea; Microdochium species, such as, for example, Microdochium nivale; Mycosphaerella species, such as, for example, Mycosphaerella graminicola and M. fijiensis; Phaeosphaeria species, such as, for example, Phaeosphaeria nodorum; Pyrenophora species, such as, for example, Pyrenophora teres; Ramularia species, such as, for example, Ramularia collo-cygni; Rhynchosporium species, such as, for example, Rhynchosporium secalis; Septoria species, such as, for example, Septoria apii; Typhula species, such as, for example, Typhula incamata; Venturia species, such as, for example, Venturia inaequalis;
root and stem diseases caused, for example, by Corticium species, such as, for example, Corticium graminearum; Fusarium species, such as, for example, Fusarium oxysporum; Gaeumannomyces species, such as, for example, Gaeumannomyces graminis; Rhizoctonia species, such as, for example Rhizoctonia solani; Tapesia species, such as, for example, Tapesia acuformis; Thielaviopsis species, such as, for example, Thielaviopsis basicola;
ear and panicle diseases (including maize cobs) caused, for example, by Alternaria species, such as, for example, Alternaria spp.; Aspergillus species, such as, for example, Aspergillus flavus; Cladosporium species, such as, for example, Cladosporium cladosporioides; Claviceps species, such as, for example, Claviceps purpurea; Fusarium species, such as, for example, Fusarium culmorum; Gibberella species, such as, for example, Gibberella zeae; Monographella species, such as, for example, Monographella nivalis; Septoria species, such as, for example, Septoria nodorum;
diseases caused by smut fungi, such as, for example, Sphacelotheca species, such as, for example, Sphacelotheca reiliana; Tilletia species, such as, for example, Tilletia caries, T. controversa; Urocystis species, such as, for example, Urocystis occulta; Ustilago species, such as, for example, Ustilago nuda, U. nuda tritici; fruit rot caused, for example, by Aspergillus species, such as, for example, Aspergillus flavus; Botrytis species, such as, for example, Botrytis cinerea; Penicillium species, such as, for example, Penicillium expansum and P. purpurogenum; Sclerotinia species, such as, for example, Sclerotinia sclerotiorum;
Verticilium species, such as, for example, Verticilium alboatrum; seed- and soil-borne rot and wilt diseases, and also diseases of seedlings, caused, for example, by Fusarium species, such as, for example, Fusarium culmorum; Phytophthora species, such as, for example, Phytophthora cactorum; Pythium species, such as, for example, Pythium ultimum; Rhizoctonia species, such as, for example, Rhizoctonia solani; Sclerotium species, such as, for example, Sclerotium rolfsii;
cancerous diseases, galls and witches' broom caused, for example, by Nectria species, such as, for example, Nectria galligena;
wilt diseases caused, for example, by Monilinia species, such as, for example, Monilinia laxa;
deformations of leaves, flowers and fruits caused, for example, by Taphrina species, such as, for example, Taphrina deformans;
degenerative diseases of woody plants caused, for example, by Esca species, such as, for example, Phaeomoniella chlamydospora and Phaeoacremonium aleophilum and Fomitiporia mediterranea;
diseases of flowers and seeds caused, for example, by Botrytis species, such as, for example, Botrytis cinerea;
diseases of plant tubers caused, for example, by Rhizoctonia species, such as, for example, Rhizoctonia solani; Helminthosporium species, such as, for example, Helminthosporium solani;
diseases caused by bacterial pathogens, such as, for example, Xanthomonas species, such as, for example, Xanthomonas campestris pv. oryzae; Pseudomonas species, such as, for example, Pseudomonas syringae pv. lachrymans; Erwinia species, such as, for example, Erwinia amylovora.

Preference is given to controlling the following diseases of soya beans:

Fungal diseases on leaves, stems, pods and seeds caused, for example, by alternaria leaf spot (Alternaria spec. atrans tenuissima), anthracnose (Colletotrichum gloeosporoides dematium var. truncatum), brown spot (Septoria glycines), cercospora leaf spot and blight (Cercospora kikuchii), choanephora leaf blight (Choanephora infundibulifera trispora (Syn.)), dactuliophora leaf spot (Dactuliophora glycines), downy mildew (Peronospora manshurica), drechslera blight (Drechslera glycini), frogeye leaf spot (Cercospora sojina), leptosphaerulina leaf spot (Leptosphaerulina trifolii), phyllostica leaf spot (Phyllosticta sojaecola), pod and stem blight (Phomopsis sojae), powdery mildew (Microsphaera diffusa), pyrenochaeta leaf spot (Pyrenochaeta glycines), rhizoctonia aerial, foliage, and web blight (Rhizoctonia solani), rust (Phakopsora pachyrhizi Phakopsora meibomiae), scab (Sphaceloma glycines), stemphylium leaf blight (Stemphylium botryosum), target spot (Corynespora cassiicola).

Fungal diseases on roots and the stem base caused, for example, by black root rot (Calonectria crotalariae), charcoal rot (Macrophomina phaseolina), fusarium blight or wilt, root rot, and pod and collar rot (Fusarium oxysporum, Fusarium orthoceras, Fusarium semitectum, Fusarium equiseti), mycoleptodiscus root rot (Mycoleptodiscus terrestris), neocosmospora (Neocosmospora vasinfecta), pod and stem blight (Diaporthe phaseolorum), stem canker (Diaporthe phaseolorum var. caulivora), phytophthora rot (Phytophthora megasperma), brown stem rot (Phialophora gregata), pythium rot (Pythium aphanidermatum, Pythium irregulare, Pythium debaryanum, Pythium myriotylum, Pythium ultimum), rhizoctonia root rot, stem decay, and damping off (Rhizoctonia solani), sclerotinia stem decay (Sclerotinia sclerotiorum), sclerotinia southern blight (Sclerotinia rolfsii), thielaviopsis root rot (Thielaviopsis basicola).

Organisms which can bring about degradation or modification of the industrial materials and which may be mentioned are fungi. The active compounds according to the invention preferably act against fungi, in particular moulds, wood-discoloring and wood-destroying fungi (Basidiomycetes). Fungi of the following genera may be mentioned as examples: Alternaria, such as Alternaria tenuis; Aspergillus, such as Aspergillus niger; Chaetomium, such as Chaetomium globosum; Coniophora, such as Coniophora puetana; Lentinus, such as Lentinus tigrinus; Penicillium, such as Penicillium glaucum; Polyporus, such as Polyporus versicolor; Aureobasidium, such as Aureobasidium pullulans; Sclerophoma, such as Sclerophoma pityophila; Trichoderma, such as Trichoderma viride.

In addition, the active compounds according to the invention also have very good antimycotic activity. They have a very broad antimycotic activity spectrum, in particular against dermatophytes and yeasts, moulds and diphasic fungi, (for example against Candida species, such as Candida albicans, Candida glabrata), and Epidermophyton floccosum, Aspergillus species, such as Aspergillus niger and Aspergillus fumigatus, Trichophyton species, such as Trichophyton mentagrophytes, Microsporon species such as Microsporon canis and audouinii. The list of these fungi by no means constitutes a restriction of the mycotic spectrum covered, and is merely of illustrative character.

When using the active compounds according to the invention as fungicides, the application rates can be varied within a relatively wide range, depending on the kind of application. The application rate of the active compounds according to the invention is
in the case of treatment of plant parts, for example leaves: from 0.1 to 10 000 g/ha, preferably from 10 to 1000 g/ha, more preferably from 50 to 300 g/ha (in the case of application by watering or dripping, it is even possible to reduce the application rate, especially when inert substrates such as rockwool or perlite are used);
in the case of seed treatment: from 2 to 200 g per 100 kg of seed, preferably from 3 to 150 g per 100 kg of seed, more preferably from 2.5 to 25 g per 100 kg of seed, even more preferably from 2.5 to 12.5 g per 100 kg of seed;
in the case of soil treatment: from 0.1 to 10 000 g/ha, preferably from 1 to 5000 g/ha.

These application rates are mentioned only by way of example and are not limiting in the sense of the invention.

The active compounds or compositions according to the invention can thus be employed for protecting plants for a certain period of time after treatment against attack by the pathogens mentioned. The period for which protection is provided extends generally for 1 to 28 days, preferably for 1 to 14 days, particularly preferably for 1 to 10 days, very particularly preferably for 1 to 7 days after the treatment of the plants with the active compounds, or for up to 200 days after a seed treatment.

In addition, by the treatment according to the invention it is possible to reduce the mycotoxin content in the harvested material and the foodstuffs and feedstuffs prepared therefrom. Particular, but not exclusive, mention may be made here of the following mycotoxins: deoxynivalenol (DON), nivalenol, 15-Ac-DON, 3-Ac-DON, T2- and HT2-toxin, fumonisins, zearalenon, moniliformin, fusarin, diaceotoxyscirpenol (DAS), beauvericin, enniatin, fusaroproliferin, fusarenol, ochratoxins, patulin, ergot alkaloids and aflatoxins produced, for example, by the following fungi: Fusarium spec., such as Fusarium acuminatum, F. avenaceum, F. crookwellense, F. culmorum, F. graminearum (Gibberella zeae), F. equiseti, F. fujikoroi, F. musarum, F. oxysporum, F. proliferatum, F. poae, F. pseudograminearum, F. sambucinum, F. scirpi, F. semitectum, F. solani, F. sporotrichoides, F. langsethiae, F. subglutinans, F. tricinctum, F. verticillioides, inter alia, and also by Aspergillus spec., Penicillium spec., Claviceps purpurea, Stachybotrys spec., inter alia.

The plants listed can be treated according to the invention in a particularly advantageous manner with the compounds of the formula (I) or the compositions according to the invention. The preferred ranges stated above for the active compounds or compositions also apply to the treatment of these plants. Particular emphasis is given to the treatment of plants with the compounds or compositions specifically mentioned in the present text.

### PREPARATION EXAMPLES

### Preparation of Compound No. I-4

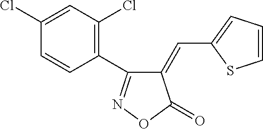

A solution of 10.0 g (40.5 mmol) of ethyl 2,4-dichlorobenzoylacetate, 5.0 g (71.6 mmol) of hydroxylammonium chloride and 3.00 g (21.7 mmol) of potassium carbonate in 100 ml of ethanol was heated at 40° C. for 3-4 h. The solvent was then distilled off, water was added, the mixture was acidified with diluted hydrochloric acid and the resulting precipitated solid was filtered off with suction, washed with ethanol and dried under reduced pressure. This gave, in a purity of 92% (HPLC), 3.70 g (16.1 mmol, 40% of theory) of 3-(2,4-dichlorophenyl)isoxazol-5(4H)-one having the log P (HCOOH)=2.44.

A solution of 1.00 g (43.5 mmol) of 3-(2,4-dichlorophenyl)isoxazol-5(4H)-one, 281 mg (2.09 mmol) of thiophene-2-carbaldehyde and 100 mg (1.15 mmol) of morpholine in 50 ml of trichloromethane was then stirred at room temperature for 24 h. The solvent was then distilled off, and the solidified residue was washed with a little ethanol and dried under reduced pressure. This gave, in a purity of 92% (HPLC), 1.00 g (30.8 mmol, 71% of theory) of 3-(2,4-dichlorophenyl)-4-(2-thienylmethylene)isoxazol-5(4H)-one having the log P (HCOOH)=3.72.

### Preparation of Compound No. I-27

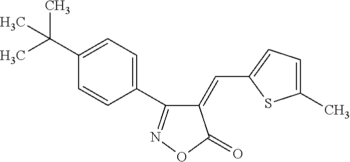

A solution of 10.0 g (40.3 mmol) of ethyl 4-tert-butylbenzoylacetate, 3.0 g (43.2 mmol) of hydroxylammonium chloride and 3.00 g (35.7 mmol) of sodium carbonate in 100 ml of ethanol was heated at reflux for 4 h. The solvent was then distilled off, water was added, the mixture was acidified with diluted hydrochloric acid and the resulting precipitated solid was filtered off with suction, washed with ethanol and dried under reduced pressure. This gave, in a purity of 99% (HPLC), 6.10 g (28.1 mmol, 70% of theory) of 3-(4-tert-butylphenyl)isoxazol-5(4H)-one having the log P(HCOOH)=3.10.

A solution of 0.50 g (23.0 mmol) of 3-(4-tert-butylphenyl)isoxazol-5(4H)-one, 290 mg (23.0 mmol) of 5-methylthiophene-2-carbaldehyde and 100 mg (1.15 mmol) of morpholine in 50 ml of trichloromethane was then stirred at room temperature for 16 h. The solvent was then distilled off, and the solidified residue was washed with a little ethanol and dried under reduced pressure. This gave, in a purity of 97% (HPLC), 0.38 g (11.7 mmol, 51% of theory) of 3-(4-tert-butylphenyl)-4-(2-(5-methylthienyl)methylene)isoxazol-5(4H)-one having the log P(HCOOH)=4.70.

### Preparation of Compound No. I-37

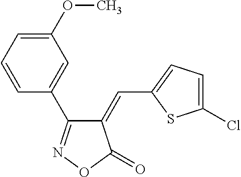

A solution of 22.0 g (99.0 mmol) of methyl 3-methoxybenzoylacetate, 13.8 g (198 mmol) of hydroxylammonium chloride and 16.6 g (198 mmol) of sodium bicarbonate in 150 ml of ethanol was heated at reflux for 5 h. The solvent was then distilled off, water was added, the mixture was acidified with diluted hydrochloric acid and the resulting precipitated solid was filtered off with suction, washed with ethanol and dried under reduced pressure. This gave, in a purity of 96% (HPLC), 11.1 g (58.1 mmol, 59% of theory) of 3-(3-methoxyphenyl)isoxazol-5(4H)-one having the log P(HCOOH)=4.03.

A solution of 0.50 g (2.62 mmol) of 3-(3-methoxyphenyl)isoxazol-5(4H)-one, 383 mg (2.62 mmol) of 2-chlorothiophene-2-carbaldehyde and 100 mg (1.15 mmol) of morpholine in 50 ml of chloroform was then stirred at room temperature overnight. The solvent was then distilled off, and the solidified residue was washed with a little ethanol and dried under reduced pressure. This gave, in a purity of 100% (HPLC), 470 mg (1.46 mmol, 56% of theory) of 3-(3-methoxyphenyl)-4-(2-(5-chlorothienyl)methylene)isoxazol-5(4H)-one having the log P (HCOOH)=3.74.

The compounds of the formula (I) listed in Table I below are also obtained by the methods given above.

TABLE I

| | |
|---|---|
| | (I) |
| 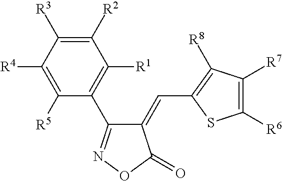 | |

| No. | R¹ | R² | R³ | R⁴ | R⁵ | R⁶ | R⁷ | R⁸ | log P | Group |
|---|---|---|---|---|---|---|---|---|---|---|
| I-1 | H | H | H | H | H | Br | H | H | 3.71 | 4 |
| I-2 | H | H | F | H | H | H | H | H | 3.01 | 3 |
| I-3 | H | Cl | H | H | H | H | H | H | 3.40 | 2 |
| I-4 | Cl | H | Cl | H | H | H | H | H | 3.72 | 1 |
| I-5 | H | H | Cl | H | H | H | H | H | 3.44 | 3 |
| I-6 | Cl | H | H | H | H | H | H | H | 3.17 | 1 |
| I-7 | H | Cl | Cl | H | H | H | H | H | 3.89 | 2 |
| I-8 | Cl | H | H | H | H | Me | H | H | 3.23 | 1 |
| I-9 | Cl | H | H | H | H | Cl | H | H | 3.90 | 1 |
| I-10 | Cl | H | H | H | H | Et | Br | H | 3.87 | 1 |
| I-11 | Cl | H | H | H | H | H | Br | H | 3.92 | 1 |
| I-12 | Cl | H | H | H | H | Me | Br | H | 4.73 | 1 |
| I-13 | Cl | H | H | H | H | MSM | Br | H | 4.29 | 1 |
| I-14 | Cl | H | H | H | H | H | Ph | H | 4.36 | 1 |
| I-15 | Cl | H | H | H | H | Ph | H | H | 4.42 | 1 |
| I-16 | Cl | H | H | H | H | H | H | MeS | 3.59 | 1 |
| I-17 | Cl | H | H | H | H | Br | Br | H | 4.64 | 1 |
| I-18 | Cl | H | H | H | H | H | H | Me | 3.38 | 1 |
| I-19 | Cl | H | H | H | H | Me | H | H | 3.44 | 1 |
| I-20 | Cl | H | H | H | H | H | H | MOM | 3.20 | 1 |
| I-21 | CF₃ | H | H | H | H | H | H | H | 3.03 | 1 |
| I-22 | F | H | H | H | H | H | H | H | 2.95 | 1 |
| I-23 | Me | H | H | H | H | H | H | H | 2.98 | 1 |
| I-24 | H | NO₂ | H | H | H | H | H | H | 2.94 | 2 |
| I-25 | H | H | Cl | H | H | Me | H | H | 3.79 | 3 |
| I-26 | H | H | Cl | H | H | H | H | Me | 3.70 | 3 |
| I-27 | H | H | tBu | H | H | Me | H | H | 4.70 | 3 |
| I-28 | H | H | tBu | H | H | H | H | Me | 4.58 | 3 |
| I-29 | H | H | tBu | H | H | H | H | H | 4.39 | 3 |
| I-30 | H | H | F | H | H | H | Br | H | 3.76 | 3 |
| I-31 | H | H | Cl | H | H | H | Br | H | 4.19 | 3 |
| I-32 | H | H | F | H | H | H | H | Me | 3.26 | 3 |
| I-33 | H | H | F | H | H | Me | H | H | 3.34 | 3 |
| I-34 | H | Cl | H | Cl | H | H | H | H | 4.02 | 2 |
| I-35 | Cl | H | H | Cl | H | H | H | H | 3.64 | 1 |
| I-36 | H | H | OCF₃ | H | H | Cl | H | H | 4.42 | 3 |
| I-37 | H | OMe | H | H | H | Cl | H | H | 3.74 | 4 |
| I-38 | H | H | Me | H | H | H | H | Br | 4.25 | 6 |
| I-39 | H | H | OMe | H | H | H | H | Br | 3.65 | 6 |
| I-40 | H | H | tBu | H | H | Cl | H | H | 5.08 | 3 |
| I-41 | H | H | F | H | H | Cl | H | H | 3.72 | 3 |
| I-42 | H | H | Me | H | H | Cl | H | H | 4.05 | 4 |
| I-43 | H | H | OMe | H | H | Cl | H | H | 3.70 | 4 |
| I-44 | H | H | OMe | H | H | Br | Br | H | 4.45 | 4 |
| I-45 | H | H | Cl | H | H | Br | Br | H | 4.88 | 3 |
| I-46 | 11 | H | Cl | H | H | Cl | H | H | 4.17 | 3 |
| I-47 | H | H | F | H | H | Br | H | H | 3.78 | 3 |
| I-48 | H | H | Me | H | H | Br | H | H | 4.12 | 4 |
| I-49 | H | H | OMe | H | H | Br | H | H | 3.77 | 4 |
| I-50 | H | H | Cl | H | H | Br | H | H | 4.21 | 3 |
| I-51 | H | H | Cl | H | H | NO₂ | H | H | 3.70 | 3 |
| I-52 | H | OMe | H | H | H | H | Ph | H | 4.27 | 5 |
| I-53 | H | H | tBu | H | H | NO₂ | H | H | 4.52 | 3 |
| I-54 | H | H | F | H | H | NO₂ | H | H | 3.32 | 3 |
| I-55 | H | H | OCF₃ | H | H | H | H | H | 3.80 | 3 |
| I-56 | H | H | CF₃ | H | H | H | H | H | 3.68 | 3 |
| I-57 | H | CF₃ | H | H | H | H | H | H | 3.61 | 2 |
| I-58 | H | H | OEt | H | H | H | H | H | 3.41 | 3 |
| I-59 | F | H | H | H | F | H | H | H | 2.97 | 1 |
| I-60 | H | H | F | H | H | H | H | Br | 3.74 | 3 |
| I-61 | H | H | Cl | H | H | H | H | Br | 4.23 | 3 |
| I-62 | H | H | OMe | H | H | H | Ph | H | 4.23 | 5 |
| I-63 | H | H | F | H | H | Br | Br | H | 4.45 | 3 |
| I-64 | H | H | Me | H | H | Br | Br | H | 4.84 | 4 |
| I-65 | H | H | Cl | H | H | H | Ph | H | 4.70 | 3 |
| I-66 | H | H | Ph | H | H | H | H | H | 4.16 | 3 |
| I-67 | H | H | F | H | H | H | Ph | H | 4.27 | 3 |
| I-68 | H | OMe | H | H | H | Br | H | H | 3.79 | 4 |
| I-69 | H | H | Me | H | H | H | Ph | H | 5.30 | 5 |
| I-70 | F | H | H | H | F | H | Ph | H | 4.16 | 1 |
| I-71 | H | H | CF₃ | H | H | Cl | H | H | 4.37 | 3 |
| I-72 | F | H | H | H | F | Br | H | H | 3.73 | 1 |
| I-73 | F | H | H | H | F | H | H | Me | 3.17 | 1 |
| I-74 | H | OMe | H | H | H | H | Fl | Br | 3.73 | 6 |
| I-75 | H | H | Ph | H | H | Br | H | H | 4.88 | 3 |
| I-76 | F | H | H | H | F | NO₂ | H | H | 3.25 | 1 |
| I-77 | F | H | H | H | F | H | Br | H | 3.69 | 1 |
| I-78 | F | H | H | H | F | Me | H | H | 3.25 | 1 |
| I-79 | H | H | OiPr | H | H | H | H | H | 3.73 | 3 |
| I-80 | H | Me | H | H | H | H | H | H | 3.36 | 2 |
| I-81 | H | H | OEt | H | H | Me | H | H | 3.70 | 3 |
| I-82 | H | H | OEt | H | H | H | H | Me | 3.61 | 3 |
| I-83 | H | H | OEt | H | H | Br | H | H | 4.20 | 3 |
| I-84 | H | Me | H | H | H | Br | H | H | 4.14 | 2 |
| I-85 | H | Me | H | H | H | Me | H | H | 3.67 | 2 |
| I-86 | H | Me | H | H | H | H | H | Me | 3.60 | 2 |
| I-87 | H | Me | H | Me | H | Me | H | H | 4.08 | 2 |
| I-88 | H | Me | H | Me | H | H | H | Me | 4.03 | 2 |
| I-89 | H | Me | H | Me | H | H | H | H | 3.78 | 2 |
| I-90 | Me | H | Me | H | H | H | H | H | 3.60 | 1 |
| I-91 | H | CF₃ | H | H | H | H | H | Me | 3.82 | 2 |
| I-92 | H | Me | Me | H | H | H | H | H | 3.73 | 2 |
| I-93 | H | H | tBu | H | H | Br | H | H | 5.13 | 3 |
| I-94 | H | H | | H | H | H | H | H | 4.14 | 3 |
| I-95 | H | H | | H | H | H | H | Me | 4.33 | 3 |
| I-96 | H | H | | H | H | H | H | H | 4.07 | 3 |
| I-97 | Me | H | Me | H | H | H | H | Me | 3.85 | 1 |
| I-98 | Me | H | H | Me | H | H | H | H | 3.62 | 1 |
| I-99 | Cl | Cl | H | H | H | H | H | H | 3.62 | 1 |
| I-100 | H | F | H | H | H | H | H | H | 3.13 | 2 |
| I-101 | H | F | H | H | H | NO₂ | H | H | 3.41 | 2 |
| I-102 | H | F | H | H | H | H | H | Me | 3.31 | 2 |
| I-103 | Cl | H | H | H | H | Br | H | H | 3.94 | 1 |
| I-104 | Cl | H | H | H | H | NO₂ | H | H | 3.50 | 1 |
| I-105 | H | H | H | H | H | Cl | H | H | 3.76 | 4 |
| I-106 | F | H | H | H | H | Cl | H | H | 3.67 | 1 |
| I-107 | I | H | H | H | H | H | H | Me | 3.56 | 1 |
| I-108 | I | H | H | H | H | H | H | H | 3.36 | 1 |
| I-109 | H | H | H | H | H | H | H | Br | 3.69 | 6 |
| I-110 | H | H | H | H | H | H | Ph | H | 4.27 | 5 |
| I-111 | H | H | H | H | H | Br | Br | H | 4.46 | 4 |
| I-112 | OEt | H | H | H | H | H | Ph | H | 4.44 | 1 |
| I-113 | OBz | H | H | H | H | Br | H | H | 4.48 | 1 |
| I-114 | OBz | H | H | H | H | H | Ph | H | 4.80 | 1 |
| I-115 | OBz | H | H | H | H | Me | H | H | 3.98 | 1 |
| I-116 | OBz | H | H | H | H | H | H | Me | 3.89 | 1 |
| I-117 | OBz | H | H | H | H | H | H | H | 3.74 | 1 |
| I-118 | OEt | H | H | H | H | Br | H | H | 4.03 | 1 |
| I-119 | OEt | H | H | H | H | H | H | H | 3.25 | 1 |
| I-120 | NO₂ | H | H | H | H | H | H | H | 2.69 | 1 |
| I-121 | OCF₃ | H | H | H | H | H | H | H | 3.43 | 1 |
| I-122 | Br | H | H | Br | H | H | H | H | 3.88 | 1 |
| I-123 | Me | Me | H | H | H | H | H | H | 3.48 | 1 |
| I-124 | Me | H | H | Me | H | H | H | Me | 3.81 | 1 |
| I-125 | Br | H | H | Br | H | H | H | Me | 4.02 | 1 |
| I-126 | F | H | H | F | H | H | H | H | 3.07 | 1 |
| I-127 | Cl | H | H | F | H | H | H | H | 3.28 | 1 |
| I-128 | Cl | H | H | Br | H | H | H | H | 3.74 | 1 |
| I-129 | Cl | H | H | F | H | Br | H | H | 4.06 | 1 |
| I-130 | Cl | H | H | F | H | H | H | Me | 3.55 | 1 |
| I-131 | Cl | H | H | Br | H | Me | H | H | 4.02 | 1 |
| I-132 | Cl | H | H | Br | H | H | H | Me | 3.97 | 1 |
| I-133 | Cl | H | H | F | H | NO₂ | H | H | 3.50 | 1 |
| I-134 | NO₂ | H | H | H | H | Br | H | H | 3.35 | 1 |
| I-135 | NO₂ | H | H | H | H | H | H | Me | 2.85 | 1 |
| I-136 | Cl | H | H | F | H | H | H | Cl | 3.95 | 1 |
| I-137 | Cl | H | H | F | H | H | Br | H | 3.95 | 1 |
| I-138 | Cl | H | H | Br | H | Cl | H | H | 4.45 | 1 |
| I-139 | F | H | H, | F | H | Cl | H | H | 3.84 | 1 |

| | | | | | | | | | |
|---|---|---|---|---|---|---|---|---|---|
| I-140 | Cl | H | H | H | H | (1Z,3Z)-buta-1,3-diene-1,4-diyl | Me | 4.31 | 1 |
| I-141 | H | H | OMe | H | H | (1Z,3Z)-buta-1,3-diene-1,4-diyl | Me | 4.09 | 4 |
| I-142 | H | H | Me | H | H | (1Z,3Z)-buta-1,3-diene-1,4-diyl | Me | 4.51 | 4 |
| I-143 | H | H | F | H | H | (1Z,3Z)-buta-1,3-diene-1,4-diyl | Me | 4.18 | 3 |
| I-144 | H | H | Cl | H | H | (1Z,3Z)-buta-1,3-diene-1,4-diyl | Me | 4.63 | 3 |
| I-145 | H | H | H | H | H | (1Z,3Z)-buta-1,3-diene-1,4-diyl | Me | 4.13 | 4 |
| I-146 | Cl | H | H | F | H | (1Z,3Z)-buta-1,3-diene-1,4-diyl | Me | 4.32 | 1 |

Me = methyl, Et = ethyl, MeS = methylsulphanyl, MOM = methoxymethyl, MSM = (methylsulphanyl)methyl, tBu = tert-butyl, Ph = phenyl, OMe = methoxy, OEt = ethoxy, OiPr = propan-2-yloxy, OPh = phenoxy, OBz = benzyloxy

Table U shows further examples of the formula (I) whose use as fungicides is claimed. They belong to group U.

TABLE U

| | | | | | | | | | (I) |
|---|---|---|---|---|---|---|---|---|---|
| 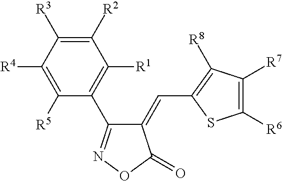 | | | | | | | | | |
| No. | R¹ | R² | R³ | R⁴ | R⁵ | R⁶ | R⁷ | R⁸ | log P |
| U-1 | H | H | H | H | H | H | H | H | 2.91 |
| U-2 | H | H | Me | H | H | H | H | H | 3.32 |
| U-3 | H | H | H | H | H | Me | H | H | 3.27 |
| U-4 | H | H | H | H | H | H | H | H | 3.41 |
| U-5 | H | H | H | H | H | H | H | H | 2.47 |
| U-6 | H | OMe | H | H | H | H | H | H | 2.90 |
| U-7 | H | H | Me | H | H | H | Br | H | 4.09 |
| U-8 | H | H | Me | H | H | H | H | Me | 3.57 |
| U-9 | H | H | OMe | H | H | Me | H | H | 3.28 |
| U-10 | H | H | Me | H | H | Me | H | H | 3.67 |
| U-11 | H | H | OMe | H | H | H | H | Me | 3.18 |
| U-12 | H | OMe | H | H | H | Me | H | H | 3.34 |
| U-13 | H | OMe | H | H | H | H | H | Me | 3.24 |
| U-14 | H | OMe | H | H | H | H | Br | H | 3.76 |
| U-15 | H | OMe | H | H | H | NO₂ | H | H | 3.38 |
| U-16 | H | H | OMe | H | H | H | Br | H | 3.71 |
| U-17 | H | H | OMe | H | H | NO₂ | H | H | 6.31 |
| U-18 | H | H | Me | H | H | NO₂ | H | H | 3.62 |
| U-19 | H | OMe | OMe | H | H | H | H | H | 3.01 |
| U-20 | H | OMe | H | OMe | H | Me | H | H | 3.48 |
| U-21 | H | OMe | H | OMe | H | H | H | H | 3.20 |
| U-22 | OMe | H | H | H | OMe | H | H | H | 2.79 |
| U-23 | H | H | NO₂ | H | H | H | H | Me | 3.18 |
| U-24 | H | H | NO₂ | H | H | Me | H | H | 3.29 |
| U-25 | OMe | H | OMe | H | H | Me | H | H | 2.97 |
| U-26 | H | OMe | OMe | H | H | H | H | Me | 3.17 |
| U-27 | OMe | H | OMe | H | H | H | H | Me | 2.81 |
| U-28 | OMe | H | OMe | H | H | H | H | H | 2.68 |
| U-29 | H | H | H | H | H | H | H | Me | 3.18 |
| U-30 | OMe | OMe | H | H | H | H | H | H | 2.97 |
| U-31 | OMe | H | H | F | H | H | H | H | 3.06 |
| U-32 | OMe | H | H | F | H | H | H | Me | 3.21 |
| U-33 | H | H | H | H | H | H | H | H | 2.91 |
| U-34 | H | H | H | H | H | Et | NO₂ | H | |
| U-35 | H | H | H | H | H | Bu | H | H | |
| U-36 | H | H | H | H | H | H | Br | H | 3.71 |
| U-37 | H | H | OMe | H | H | H | H | H | 2.95 |
| U-38 | H | H | OMe | H | H | H | H | H | 2.95 |
| U-39 | OMe | H | H | H | H | H | H | H | 2.75 |
| U-40 | H | H | NO₂ | H | H | H | H | H | 3.03 |
| U-41 | H | H | H | H | H | NO₂ | H | H | 3.27 |
| U-42 | H | H | H | H | H | Me | H | H | 3.35 |
| U-43 | H | H | H | H | H | NO₂ | H | H | |

Me = methyl, Bu = n-butyl, OMe = methoxy

The log P values were measured according to EEC directive 79/831 Annex V.A8 by HPLC (High Performance Liquid Chromatography) on reversed-phase columns (C 18), using the method below:

The determination was carried out in the acidic range at pH 2.7 using the mobile phases 0.1% aqueous formic acid and acetonitrile; linear gradient from 10% acetonitrile to 95% acetonitrile.

Calibration was carried out using unbranched alkan-2-ones (having from 3 to 16 carbon atoms) with known log P values (the log P values were determined by the retention times using linear interpolation between two successive alkanones).

The lambda max values were determined in the maxima of the chromatographic signals using the UV spectra from 200 nm to 400 nm.

The following compounds of the general formula (VII) are known from GB-A 1,074,803. Their fungicidal activities represent the fungicidal closest prior art and are compared to the fungicidal activities of the compounds according to the invention.

TABLE A

| | | | | | | (VII) |
|---|---|---|---|---|---|---|
| 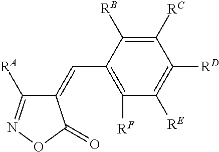 | | | | | | |
| No. | R^{A} | R^{B} | R^{C} | R^{D} | R^{E} | R^{F} |
| A-1 | Ph | H | H | H | H | H |
| A-2 | Ph | H | H | Cl | H | H |
| A-3 | Me | H | H | H | H | H |
| A-4 | Me | H | H | Me | H | H |
| A-5 | Me | H | H | OMe | H | H |
| A-6 | Ph | Cl | H | H | H | H |
| A-7 | Me | H | H | NMe₂ | H | H |

| | |
|---|---|
| A-8 | 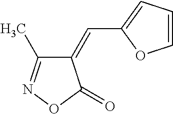 |

Me = methyl, Ph = phenyl, OMe = methoxy, NMe₂ = dimethylamino

### Use Examples

### Example A

### Blumeria Test (Wheat)/Protective

| | |
|---|---|
| Solvent: | 50 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are dusted with spores of Blumeria graminis f sp. tritici. The plants are placed in a greenhouse at a temperature of about 20° C. and a relative atmospheric humidity of about 80% to promote the development of mildew pustules. Evaluation is carried out 7 days after the inoculation. 0% means an efficacy which corresponds to that of the control, whereas an efficacy of 100% means that no infection is observed.

In this test, the compounds according to the invention I-21 and U-1 from Tables I and U show, at an active compound concentration of 1000 ppm, an efficacy of 70% or more. In this test, the active compounds from Table A exhibit the following efficacies: A-1: 29%, A-2: 29%, A-3: 57%, A-4: 43%, A-5: 29%, A-6: 29%, A-7: 0% and A-8: 29%.

### Example B

| | |
|---|---|
| Solvents: | 24.5 parts by weight of acetone |
| | 24.5 parts by weight of dimethylacetamide |
| Emulsifier: | 1 part by weight of alkylaryl polyglycol ether |

Emulsifier: 1 Part by Weight of Alkylaryl Polyglycol Ether

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are inoculated with an aqueous spore suspension of Phytophthora infestans. The plants are then placed in an incubation cabin at about 20° C. and 100% relative atmospheric humidity. Evaluation is carried out 3 days after the inoculation. 0% means an efficacy which corresponds to that of the control, whereas an efficacy of 100% means that no infection is observed.

In this test, the compounds according to the invention I-6, I-22, U-1 and U-33 from Tables I and U show, at an active compound concentration of 100 ppm, an efficacy of 70% or more. At an active compound concentration of 100 ppm, the active compounds from Table A exhibit the following efficacies: A-2: 35%, A-3: 15%, A-4: 62%, A-5: 53%, A-6: 18%, A-7: 0% and A-8: 0%.

### Example C

### Plasmopara Test (Grapevine)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are inoculated with an aqueous spore suspension of Plasmopara viticola and then remain in an incubation cabin at about 20° C. and 100% relative atmospheric humidity for 1 day. The plants are then placed in a greenhouse at about 21° C. and about 90% atmospheric humidity for 4 days. The plants are then moistened and placed in an incubation cabin for 1 day. Evaluation is carried out 6 days after the inoculation. 0% means an efficacy which corresponds to that of the control, whereas an efficacy of 100% means that no infection is observed.

In this test, the compounds according to the invention I-2, I-3, I-5, I-6, I-9, I-20, I-21, I-22, I-23, I-56, I-59, I-73, I-78, U-1, U-33, U-38 from Tables I and U show, at an active compound concentration of 100 ppm, an efficacy of 70% or more. At an active compound concentration of 100 ppm, the active compounds from Table A exhibit the following efficacies: A-2: 38%, A-3: 55%, A-4: 35%, A-5: 40%, A-6: 38%, A-7: 45%, A-8: 28%.

### Example D

### Venturia Test (Apple)/Protective

| | |
|---|---|
| Solvents: | 24.5 parts by weight of acetone |
| | 24.5 parts by weight of dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young plants are sprayed with the active compound preparation at the stated application rate. After the spray coating has dried on, the plants are inoculated with an aqueous conidia suspension of the apple scab pathogen Venturia inaegualis and then remain in an incubation cabin at about 2° C. and 100% relative atmospheric humidity for 1 day. The plants are then placed in the greenhouse at about 21° C. and a relative atmospheric humidity of about 90%. Evaluation is carried out 10 days after the inoculation. 0% means an efficacy which corresponds to that of the control, whereas an efficacy of 100% means that no infection is observed.

In this test, the compounds according to the invention I-3, I-5, I-6, I-21, I-54, I-56, I-72, I-73, I-90 from Table I show, at an active compound concentration of 100 ppm, an efficacy of 70% or more. At an active compound concentration of 100 ppm, the active compounds from Table A exhibit the following efficacies: A-2: 16%, A-3: 48%, A-4: 9%, A-5: 59%, A-6: 14%, A-7: 0% and A-8: 6%.

### Example E

### Alternaria Test (Tomato)/Protective

| | |
|---|---|
| Solvent: | 49 parts by weight of N,N-dimethylacetamide |
| Emulsifier: | 1 part by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young tomato plants are sprayed with the active compound preparation at the stated application rate. 1 day after the treatment, the plants are inoculated with a spore suspension of Alternaria solani and then remain at 100% relative humidity and 20° C. for 24 h. The plants then remain at 96% relative atmospheric humidity and a temperature of 20° C. Evaluation is carried out 7 days after the inoculation. 0% means an efficacy which corresponds to that of the control, whereas an efficacy of 100% means that no infection is observed.

In this test, the compounds according to the invention I-35, I-40, I-51, I-54, I-59, I-90, I-91, I-127, I-129, I-130, I-136, U-10, U-18, U-21, U-40 from Tables I and U show, at an active compound concentration of 500 ppm, an efficacy of 70% or more. At an active compound concentration of 500 ppm, the active compounds from Table A exhibit the following efficacies: A-1: 0%, A-2: 0%, A-3: 0%, A-4: 0%, A-5: 0%, A-6: 0%, A-7: 0% and A-8: 0%.

### Example F

### In Vitro Test for Determining the ED₅₀ Value with Leptosphaeria Nodorum

The cavities of 96-well microtitre plates are filled with 1.5 μl of a solution of the test compounds in DMSO. 150 μl of a glucose/peptone medium comprising the spores of the test fungus in a suitable concentration are introduced into each cavity. The test compounds in the microtitre cavities are used in concentrations of 20, 6, 2 and 0.6 ppm. The absorbance is determined photometrically at 620 nm. The microtitre plates are left at 20° C. and 85% relative atmospheric humidity for 6 days. Once the incubation time has passed, the growth of the test organisms is determined photometrically at a wavelength of 620 nm. The difference in absorbances before and after the incubation corresponds to the growth of the test fungi. Based on the difference of the absorbances at the various test concentrations and the difference of the absorbances of the untreated control, a dose/activity curve is calculated. The concentration required to inhibit fungal growth by 50% is determined and reported as ED₅₀ value (=Effective Dose which causes 50% inhibition of growth) in ppm (=mg/l).

In this test, the compounds according to the invention I-5, I-8, I-9, I-13, I-15, I-16, I-18, I-19, I-23, I-103, I-106, I-107, I-126, I-140 and U-1 from Tables I and U have an ED₅₀ values of less than 1 ppm. The active compounds from Table A have the following ED₅₀ values: A-1: >20 ppm, A-2: >20 ppm, A-3: >20 ppm, A-4: 14 ppm.

### Example G

### Pyricularia Test (Rice)/Protective

| | |
|---|---|
| Solvent: | 28.5 parts by weight of acetone |
| Emulsifier: | 1.5 parts by weight of alkylaryl polyglycol ether |

To produce a suitable preparation of active compound, 1 part by weight of active compound is mixed with the stated amounts of solvent and emulsifier, and the concentrate is diluted with water to the desired concentration. To test for protective activity, young rice plants are sprayed with the active compound preparation at the stated application rate. 1 day after the treatment, the plants are inoculated with an aqueous spore suspension of Pyricularia oryzae. The plants are then placed in a greenhouse at 100% relative atmospheric humidity and a temperature of 25° C. for one day. Evaluation is carried out 7 days after the inoculation. 0% means an efficacy which corresponds to that of the control, whereas an efficacy of 100% means that no infection is observed.

In this test, the compounds according to the invention I-6, I-98 and I-99 from Table I exhibit, at an active compound concentration of 250 ppm, an efficacy of 80% or more.

### Example H

### Phaedon Test (Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. Discs of Chinese cabbage (Brassica pekinensis) are sprayed with an active compound preparation of the desired concentration and, after drying, populated with larvae of the mustard beetle (Phaedon cochleariae). After the desired period of time, the effect in % is determined. 100% means that all beetle larvae have been killed; 0% means that none of the beetle larvae have been killed.

In this test, for example, the compounds according to the invention I-22, I-29, I-33 and I-55 from Table I exhibit, at an application rate of 500 g/ha, an activity of >80%.

### Example J Spodoptera Frugiperda Test (Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. Discs of maize leaves (Zea mays) are sprayed with an active compound preparation of the desired concentration and, after drying, populated with caterpillars of the armyworm (Spodoptera frugiperda). After the desired period of time, the effect in % is determined. 100% means that all caterpillars have been killed; 0% means that none of the caterpillars have been killed.

In this test, for example, the compounds according to the invention I-5, I-29, I-30, I-32, I-33, I-43, I-55, I-56, I-58, I-79, U-1, U-2, U-9, U-10, U-12, U-33, U-37, U-38 and U-39 from Tables I and U exhibit, at an application rate of 500 g/ha, an activity of 80%.

### Example I

### Heliotis Virescens Test (Spray Treatment)

| | |
|---|---|
| Solvents: | 78 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvent and emulsifier, and the concentrate is diluted with emulsifier-containing water to the desired concentration. Soya bean leaves (Glycine max) are sprayed with an active compound preparation of the desired concentration and, after drying, populated with larvae of the cotton bollworm (Heliothis virescens). After the desired period of time, the effect in % is determined. 100% means that all of the eggs have been killed; 0% means that none of the eggs have been killed.

In this test, for example, the compounds according to the invention I-5 and U-37 from Tables I and U exhibit, at an application rate of 500 g/ha, an activity of ≧80%.

1. A 3-aryl-4-(2-thienylmethylene)isoxazol-5(4H)-one of formula (I) 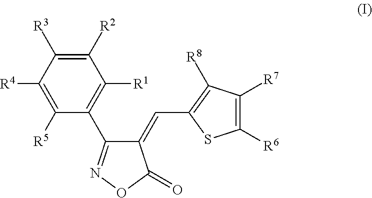
in which
(1) group 1:
R¹ represents A, but not hydrogen, hydroxyl or methoxy,
R² to R⁵ independently of one another represent A, where furthermore in each case two adjacent radicals R¹ to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent, and
R⁶ to R⁸ independently of one another represent A,
or
(2) group 2:
R¹ represents hydrogen or methoxy,
R² represents A, but not hydrogen, methoxy, nitro or dimethylcarbamothioylamino [NHC(S)NMe₂],
R³ to R⁵ independently of one another represent A, where furthermore in each case two adjacent radicals R² to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent, and
R⁶ to R⁸ independently of one another represent A,
or
(3) group 3:
R¹ represents hydrogen or methoxy,
R² represents hydrogen, methoxy, nitro or dimethylcarbamothioylamino [NHC(S)NMe₂],
R³ represents A, but does not represent hydrogen, nitro, C₁-C₃-alkoxy, amino, CO₂H or methyl,
R⁴ to R⁵ independently of one another represent A, where furthermore in each case two adjacent radicals R³ to R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent, and
R⁶ to R⁸ independently of one another represent A,
or
(4) group 4:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ to R⁵ independently of one another represent A, where furthermore R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents A, but not hydrogen, nitro, dialkylamino, C₁-C₈-alkyl, hetaryl or morpholin-4-yl, and
R⁷ and R⁸ independently of one another represent A,
or
(5) group 5:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ to R⁵ independently of one another represent A, where furthermore the radicals R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents hydrogen, nitro, dialkylamino, C₁-C₈-alkyl, hetaryl or morpholin-4-yl,
R⁷ represents A, but not hydrogen, bromine or nitro, and
R⁸ represents A,
or
(6) group 6:
R¹ represents hydrogen or methoxy,
R² represents hydrogen, methoxy, nitro or dimethylcarbamothioylamino [NHC(S)NMe₂],
R³ represents hydrogen, nitro, C₁-C₃-alkoxy, amino or methyl,
R⁴ and R⁵ independently of one another represent A, where furthermore R⁴ and R⁵ together with the carbon atoms to which they are attached may form an unsubstituted or substituted unsaturated or fully or partially saturated heterocyclic or carbocyclic 5- to 7-membered ring which, depending on the ring size, may contain up to 3 further nitrogen atoms or alternatively up to 2 further heteroatoms selected from the group consisting of N, O and S, where two oxygen atoms are not adjacent,
R⁶ represents hydrogen, nitro, dialkylamino, C₁-C₈-alkyl, hetaryl or morpholin-4-yl,
R⁷ represents hydrogen, bromine or nitro, and
R⁸ represents A, but not hydrogen or methyl,
and
A represents hydrogen, halogen, cyano, nitro, OR⁹, SR⁹, SOR⁹, SO₂R⁹, SO₂NR⁹R¹⁰, COR^{S}, C═N—OR⁹, CSR⁹, NR⁹CO₂R¹⁰, NR⁹C(O)SR¹⁰, NR⁹C(S)OR¹⁰, NR⁹R¹⁰, NR⁹COR¹⁰, NR⁹CSR¹⁰, NR⁹SO₂R¹⁰, OCONR⁹R¹⁰, OCSNR⁹R¹⁰, NR⁹C(O)NR⁹R¹⁰, NR⁹C(S)NR⁹R¹⁰, O(CO)R⁹, O(CS)R⁹, CONR⁹R¹⁰, CSNR⁹R¹⁰, CO₂R⁹, C(O)SR⁹, C(S)OR⁹, (CH₂)ₘOR⁹, (CH₂)ₘSR⁹, (CH₂)ₘNR⁹R¹⁰, (CH₂)ₘCO₂R¹⁰, (CH₂)ₘNR⁹CO₂R¹⁰, C₁-C₈-alkyl, C₂-C₈-alkenyl, C₂-C₈-alkynyl, C₁-C₈-haloalkyl, C₃-C₈-cycloalkyl, C₃-C₈-cycloalkenyl, tri-C₁-C₄-alkyl-silyl, phenyl-(di-C₁-C₄-alkyl)-silyl, substituted or unsubstituted phenyl or hetaryl or heterocyclyl, substituted or unsubstituted benzyl or —CH₂-hetaryl,
R⁹ and R¹⁰ independently of one another represent hydrogen, unsubstituted or substituted C₁-C₈-alkyl, C₂-C₈-alkenyl, C₂-C₈-alkynyl, C₁-C₈-haloalkyl, C₃-C₈-cycloalkyl, tri-C₁-C₄-alkyl-silyl, in each case substituted or unsubstituted aryl, —CH₂-aryl, hetaryl or —CH₂-hetaryl or heterocyclyl, where the two radicals R⁹ and R¹⁰ together with this grouping to which they are attached form a 3- to 7-membered unsubstituted or substituted saturated or unsaturated cycle which, depending on the ring size, may contain up to 3 further nitrogen atoms or alternatively up to 2 further heteroatoms selected from the group consisting of N, O and S, where two oxygen atoms are not adjacent,
m represents the number 1, 2, 3, 4, 5, 6, 7 or 8,
and the agrochemically active salts thereof.

2-3. (canceled)

4. A method for controlling unwanted microorganisms and insects in crop protection and in the protection of materials, comprising applying the 3-aryl-4-(2-thienylmethylene)isoxazol-5(4H)-one of the formula (I) according to claim 1 to the phytopathogenic harmful fungi and/or their habitat.

5. A composition for controlling unwanted microorganisms and insects, comprising at least one 3-aryl-4-(2-thienylmethylene)isoxazol-5(4H)-one of formula (I) according to claim 1, in addition to extenders and/or surfactants.

6. A composition according to claim 5, comprising at least one further active compound selected from the group consisting of insecticides, attractants, sterilants, bactericides, acaricides, nematicides, fungicides, growth regulators, herbicides, fertilizers, safeners and semiochemicals.

7. A process for preparing compositions for controlling unwanted microorganisms, comprising mixing the 3-aryl-4-(2-thienylmethylene)isoxazol-5(4H)-one of formula (I) according to claim 1 with extenders and/or surfactants.

8-10. (canceled)

11. A process for preparing the 3-aryl-4-(2-thienylmethylene)isoxazol-5(4H)-one of formula (I) according to claim 1, characterized in that a 3-arylisoxazol-5(4H)-one of the formula (V) 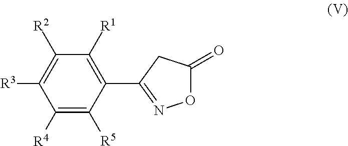
in which R¹, R², R³, R⁴ and R⁵ have the meanings given in claim 1 is reacted with a thiophene-2-carbaldehyde of formula (VI) 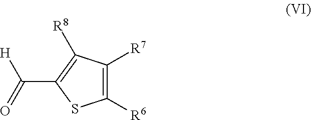
R⁶, R⁷ and R⁸ have the meanings given in claim 1,
if appropriate in the presence of a base and a diluent.

12. A method of treating transgenic plants, comprising applying the 3-aryl-4-(2-thienylmethylene)isoxazol-5(4H)-one of formula (I) according to claim 1 to the plant or plant parts.

13. A method of treating seed or transgenic seed, comprising applying the 3-aryl-4-(2-thienylmethylene)isoxazol-5(4H)-one of formula (I) according to claim 1 to the seed or transgenic seed.
